(19) United States
(12) Patent Application Publication
(10) Pub. No.: US 2026/0348855 A1
(43) Pub. Date: Nov. 5, 2026

(54) Product Formulation in Biological Manufacturing

(71) Applicant: Genzyme Corporation, Cambridge, MA (US)

(72) Inventors: Rita Barral, Cambridge, MA (US); Michael Coolbaugh, Cambridge, MA (US); Sushmitha Krishnan, Cambridge, MA (US)

(21) Appl. No.: 19/118,621

(22) PCT Filed: Oct. 6, 2023
(86) PCT No.: PCT/US2023/034676

§ 371 Date: Apr. 4, 2025

Related U.S. Application Data
(60) Provisional application No. 63/414,452, filed on Oct. 7, 2022.

(51) Int. Cl. G01N 21/65; B01F 35/21; B01F 35/22; B01F 35/82; G01N 21/3577; G01N 21/41; G01N 21/552; G01N 21/84; G01N 21/85

(52) U.S. Cl. CPC G01N 21/65; B01F 35/21; B01F 35/22; B01F 35/82; G01N 21/3577; G01N 21/41; G01N 21/552; G01N 21/85; G01N 2021/8416

(57) ABSTRACT

Methods for combining solutions include receiving a flowing first solution that includes a biological product and directing the flowing first solution along a flow path, where the flow path includes a dilution location at which a dilution apparatus is in fluid communication with the flow path; introducing a second solution into the flowing first solution at the dilution location to form a flowing third solution; measuring the biological product in the flowing first solution at a location upstream from the dilution location; measuring the biological product in the flowing third solution at a location downstream from the dilution location; determining a relative relationship between measured values of the biological product or values derived from measured values of the biological product at the upstream and downstream locations; and adjusting a flow rate of at least one of the first solution and the second solution based on the relative relationship.

### CROSS-REFERENCE TO RELATED APPLICATIONS

This application claims priority to U.S. Provisional Patent Application No. 63/414,452, filed on Oct. 7, 2022, the entire contents of which are incorporated herein by reference.

### TECHNICAL FIELD

This disclosure relates to generation of product formulations in a variety of manufacturing processes, including continuous biomanufacturing.

### BACKGROUND

Continuous biomanufacturing processes can be used to generate a wide variety of therapeutically effective products. Typically, such products are obtained in solution after generation in a bioreactor, and optionally after one or more purification or other steps have occurred. Final products are then formulated according to established specifications, and the formulation typically involves adjusting the concentration of a product, and optionally other components, in solution to match previously established specifications. Excipient solutions can be used for the purpose of adjusting the product solution to match specifications.

### SUMMARY

Certain existing batch product formulation processes use volumetric, flow-based approaches to adding excipient solutions. Typically, such processes use a single flow meter or other volume-measuring device to control the rate of addition of an excipient solution in a formulation step. In contrast, the present disclosure features methods and systems in which multiple measurements of a biomanufacturing process solution are performed. In particular, measurements are performed both upstream and downstream from a location along the solution's flow path, and the upstream and downstream measurements are used to control excipient solution addition to ensure that the final product formulation is aligned with previously established specifications for one or more products in the process solution. By using both upstream and downstream measurements, improved product quality can be achieved, and in particular, the methods and systems described herein are compatible with a variety of product purification steps which can be difficult to implement in conventional product formulation workflows due to the relatively small volumetric flow rates of product-containing solutions, and time-dependent variations in the composition of product-containing solutions.

In a first aspect, the disclosure features methods that include receiving a flowing first solution including a biological product and directing the flowing first solution along a flow path, where the flow path includes a dilution location at which a dilution apparatus is in fluid communication with the flow path; introducing a second solution into the flowing first solution at the dilution location to form a flowing third solution; measuring the biological product in the flowing first solution at a location upstream from the dilution location; measuring the biological product in the flowing third solution at a location downstream from the dilution location; determining a relative relationship between measured values of the biological product or values derived from measured values of the biological product at the upstream and downstream locations; and adjusting a flow rate of at least one of the first solution and the second solution based on the relative relationship.

Embodiments of the methods can include any one or more of the following features.

Measuring the biological product at the upstream location can include obtaining a measured value of a parameter of the first solution. The measured value of the parameter can be a refractive index of the first solution and/or a conductivity of the first solution and/or an absorbance of the first solution and/or a transmittance of the first solution and/or a reflectance of the first solution and/or a concentration of the biological product in the first solution.

The measured value of the parameter can be measured at a single wavelength. The single wavelength can be in an ultraviolet spectral region, or a visible spectral region, or an infrared spectral region.

Measuring the biological product at the upstream location can include obtaining a plurality of measured values of the first solution. The plurality of measured values can correspond to spectral information for the first solution at a plurality of wavelengths. The methods can include analyzing the spectral information to determine a value derived from the spectral information. Analyzing the spectral information can include using a calibrated chemometric model to determine the value derived from the spectral information.

The value derived from the spectral information can be a concentration of the biological product in the first solution, or a quantity related to a concentration of the biological product in the first solution. The spectral information can include an infrared spectrum of the first solution and/or an ultraviolet spectrum of the first solution and/or a Raman scattering spectrum of the first solution.

The methods can include measuring attenuated total reflection of incident infrared light to obtain the infrared spectrum. The methods can include measuring the refractive index of the first solution by measuring attenuated total reflection of incident infrared light from the first solution.

Measuring the biological product at the downstream location can include obtaining a measured value of a parameter of the third solution. The measured value of the parameter of the third solution can include at least one member of the group consisting of: a refractive index of the third solution; a conductivity of the third solution; an absorbance of the third solution; a transmittance of the third solution; a reflectance of the third solution; and a concentration of the biological product in the third solution.

The measured value of the parameter of the third solution can be measured at a single wavelength. The single wavelength can include a wavelength in an ultraviolet, visible, or infrared spectral region.

Measuring the biological product at the downstream location can include obtaining a plurality of measured values of the third solution. The plurality of measured values can correspond to spectral information for the third solution at a plurality of wavelengths.

The methods can include analyzing the spectral information to determine a value derived from the spectral information. Analyzing the spectral information can include using a calibrated chemometric model to determine the value derived from the spectral information.

The value derived from the spectral information can be a concentration of the biological product in the third solution, or a quantity related to a concentration of the biological product in the third solution. The spectral information can include at least one member of the group consisting of an infrared spectrum of the third solution, an ultraviolet spectrum of the third solution, and a Raman scattering spectrum of the third solution. The methods can include measuring attenuated total reflection of incident infrared light to obtain the infrared spectrum. The methods can include measuring the refractive index of the third solution by measuring attenuated total reflection of incident infrared light from the third solution.

Measuring the biological product at the upstream location can include obtaining at least one of a measured value of a parameter of the first solution and spectral information for the first solution, measuring the biological product at the downstream location can include obtaining at least one of a measured value of a parameter of the third solution and spectral information for the third solution, and the at least one of a measured value of a parameter of the first solution and spectral information for the first solution can be measured using a different measurement technique from the at least one of a measured value of a parameter of the third solution and spectral information for the third solution. The at least one of a measured value of a parameter of the first solution and spectral information for the first solution can include a different type of information from the at least one of a measured value of a parameter of the third solution and spectral information for the third solution. The at least one of a measured value of a parameter of the first solution and spectral information for the first solution, and the at least one of a measured value of a parameter of the third solution and spectral information for the third solution, can be each independently selected from the group consisting of a refractive index; a conductivity; an absorbance; a transmittance; a reflectance; a concentration of the biological product; an infrared spectrum; an ultraviolet spectrum; and a Raman scattering spectrum.

Measuring the biological product at the upstream location can include obtaining at least one of a measured value of a parameter of the first solution and spectral information for the first solution, measuring the biological product at the downstream location can include obtaining at least one of a measured value of a parameter of the third solution and spectral information for the third solution, and the at least one of a measured value of a parameter of the first solution and spectral information for the first solution and the at least one of a measured value of a parameter of the third solution and spectral information for the third solution can be measured using a common measurement technique. The at least one of a measured value of a parameter of the first solution and spectral information for the first solution, and the at least one of a measured value of a parameter of the third solution and spectral information for the third solution, can be each independently selected from the group consisting of a refractive index; a conductivity; an absorbance; a transmittance; a reflectance; a concentration of the biological product; an infrared spectrum; an ultraviolet spectrum; and a Raman scattering spectrum.

The second solution may not include the biological product. A flow rate of the flowing first solution along the flow path can be less than 2 mL/min. (e.g., less than 1 mL/min.).

Determining a relative relationship between measured values of the biological product or values derived from the measured values of the biological product at the upstream and downstream locations can include obtaining a first value correlated with a concentration of the biological product or a quantity related to a concentration of the biological product in the first solution at the upstream location, obtaining a second value correlated with a concentration of the biological product or a quantity related to a concentration of the biological product in the third solution at the downstream location, calculating a comparative quantity based on the first and second values, and adjusting the flow rate of the at least one of the first solution and the second solution based on the comparative quantity. The comparative quantity can be a ratio of the first and second values. The comparative quantity is a mathematical function of the first and second values.

The methods can include adjusting the flow rate of the at least one of the first solution and the second solution until a value of the relative relationship is within a target range of values. The methods can include adjusting the flow rate of the at least one of the first solution and the second solution until a value of the comparative quantity is within a target range of values.

The biological product can be a protein. The protein can be an antibody, an antibody fragment, or can include a portion of an antibody. The biological product in the first solution can be a drug substance, and the third solution can be a drug product.

The biological product can be a first biological product, the dilution location can be a first dilution location, and the flow path can include a second dilution location downstream from the first dilution location, and the methods can include introducing a fourth solution into the flowing third solution at the second dilution location to form a flowing fifth solution, where the second dilution location is downstream from a location at which the first biological product is measured in the flowing third solution, measuring a second biological product in the flowing fifth solution at locations upstream and downstream from the second dilution location, determining a relative relationship between measured values of the second biological product or values derived from the measured values of the second biological product at the upstream and downstream locations, and adjusting at least one of a flow rate of the third solution and a flow rate of the fourth solution based on the relative relationship for the second biological product.

Measuring the second biological product at the location downstream from the second dilution location can include obtaining a measured value of a parameter of the fifth solution. The measured value of the parameter of the fifth solution can include at least one member of the group consisting of: a refractive index of the fifth solution; a conductivity of the fifth solution; an absorbance of the fifth solution; a transmittance of the fifth solution; a reflectance of the fifth solution; and a concentration of the second biological product in the fifth solution.

The measured value of the parameter of the fifth solution can be measured at a single wavelength. The single wavelength can include a wavelength in an ultraviolet, visible, or infrared spectral region.

Measuring the second biological product at the location downstream from the second dilution location can include obtaining a plurality of measured values of the fifth solution. The plurality of measured values can correspond to spectral information for the fifth solution at a plurality of wavelengths.

The methods can include analyzing the spectral information for the fifth solution to determine a value derived from the spectral information. Analyzing the spectral information for the fifth solution can include using a calibrated chemometric model to determine the value derived from the spectral information. The value derived from the spectral information can be a concentration of the second biological product in the fifth solution, or a quantity related to a concentration of the second biological product in the fifth solution.

The spectral information for the fifth solution can include at least one member of the group consisting of an infrared spectrum of the fifth solution, an ultraviolet spectrum of the fifth solution, and a Raman scattering spectrum of the fifth solution. The methods can include measuring attenuated total reflection of incident infrared light to obtain the infrared spectrum of the fifth solution. The methods can include measuring the refractive index of the fifth solution by measuring attenuated total reflection of incident infrared light from the fifth solution.

The biological product can be measured at the locations upstream and downstream from the dilution location by measuring different types of spectral information corresponding to the respective first and third solutions. The measured values of the second biological product or values derived from the measured values of the second biological product at the upstream and downstream locations can be of a different type.

The methods can include obtaining the measured values of the second biological product or values derived from the measured values of the second biological product at the upstream and downstream locations using different measurement techniques.

The fourth solution may not include the second biological product. The fourth solution may not include the first biological product.

The flowing first solution can be received from a purification unit of a biological manufacturing system. The purification unit can include a tangential flow filtration unit.

The measured value of the parameter of the first solution can be an osmolality of the first solution. The measured value of the parameter of the third solution can be an osmolality of the third solution. Measuring the biological product at the upstream location can include obtaining an osmolality value for the first solution, and measuring the biological product at the downstream location can include obtaining an osmolality value for the third solution.

The methods can include adjusting the flow rate of the second solution based on the relative relationship. The relative relationship can be a ratio of the osmolality values for the first and third solutions. Embodiments of the methods can also include any of the other features described herein and can include any combination of features, including combinations of features that are separately described in different embodiments, unless expressly stated otherwise.

In another aspect, the disclosure features systems that include: a flow channel including an inlet; a fluid reservoir connected to the flow channel at a dilution location; at least one flow regulator connected between at least one of the fluid reservoir and the dilution location, and the inlet and the dilution location; a first sensor positioned at an upstream location between the inlet of the flow channel and the dilution location; a second sensor positioned at a downstream location between an outlet of the flow channel and the dilution location; and a controller connected to the first and second sensors and to the regulator, where the first sensor is configured to measure a biological product in a flowing first solution that enters the inlet, where the fluid reservoir is configured to introduce a second solution into the flow channel at the dilution location to form a flowing third solution, wherein the second sensor is configured to measure the biological product the flowing third solution, and where the controller is configured to: determine a relative relationship between measured values of the biological product or values derived from measured values of the biological product at the upstream and downstream locations; and adjust the at least one regulator to control a flow rate of at least one of the first solution and the second solution based on the relative relationship.

Embodiments of the systems can include any one or more of the following features.

The first sensor can be configured to obtain a measured value of a parameter of the first solution. The first sensor can be a refractive index sensor configured to measure a refractive index of the first solution. The first sensor can be a conductivity sensor configured to measure a conductivity of the first solution. The first sensor can be an absorbance sensor configured to measure an absorbance of the first solution. The first sensor can be a transmittance sensor configured to measure a transmittance of the first solution. The first sensor can be a reflectance sensor configured to measure a reflectance of the first solution. The first sensor can be a concentration sensor configured to measure a concentration of the biological product in the first solution.

The first sensor can be configured to measure the value of the parameter at a single wavelength. The single wavelength can be in an ultraviolet spectral region or in a visible spectral region or in an infrared spectral region.

The first sensor can be configured to obtain a plurality of measured values of the first solution. The plurality of measured values can correspond to spectral information for the first solution at a plurality of wavelengths. The controller can be configured to analyze the spectral information to determine a value derived from the spectral information. The controller can be configured to analyze the spectral information by using a calibrated chemometric model to determine the value derived from the spectral information. The value derived from the spectral information can be a concentration of the biological product in the first solution, or a quantity related to a concentration of the biological product in the first solution.

The first sensor can be configured to obtain an infrared spectrum of the first solution and/or an ultraviolet spectrum of the first solution and/or a Raman scattering spectrum of the first solution. The first sensor can be configured to measure attenuated total reflection of incident infrared light to obtain the infrared spectrum. The first sensor can be configured to measure the refractive index of the first solution by measuring attenuated total reflection of incident infrared light from the first solution.

The second sensor can be configured to obtain a measured value of a parameter of the third solution. The second sensor can be configured to measure at least one member of the group consisting of a refractive index of the third solution; a conductivity of the third solution; an absorbance of the third solution; a transmittance of the third solution; a reflectance of the third solution; and a concentration of the biological product in the third solution.

The second sensor can be configured to measure the value of the parameter of the third solution at a single wavelength. The single wavelength can include a wavelength in an ultraviolet, visible, or infrared spectral region.

The second sensor can be configured to obtain a plurality of measured values of the third solution. The plurality of measured values can correspond to spectral information for the third solution at a plurality of wavelengths. The controller can be configured to analyze the spectral information to determine a value derived from the spectral information. The controller can be configured to analyze the spectral information by using a calibrated chemometric model to determine the value derived from the spectral information. The value derived from the spectral information can be a concentration of the biological product in the third solution, or a quantity related to a concentration of the biological product in the third solution.

The second sensor can be configured to obtain spectral information including at least one member of the group consisting of an infrared spectrum of the third solution, an ultraviolet spectrum of the third solution, and a Raman scattering spectrum of the third solution. The second sensor can be configured to measure attenuated total reflection of incident infrared light to obtain the infrared spectrum. The second sensor can be configured to measure the refractive index of the third solution by measuring attenuated total reflection of incident infrared light from the third solution.

The first and second sensors can be configured to measure the biological product in the first and third solutions using different measurement techniques. The at least one of a measured value of a parameter of the first solution and spectral information for the first solution can include a different type of information from the at least one of a measured value of a parameter of the third solution and spectral information for the third solution. The at least one of a measured value of a parameter of the first solution and spectral information for the first solution, and the at least one of a measured value of a parameter of the third solution and spectral information for the third solution, can be each independently selected from the group consisting of a refractive index; a conductivity; an absorbance; a transmittance; a reflectance; a concentration of the biological product; an infrared spectrum; an ultraviolet spectrum; and a Raman scattering spectrum.

The first and second sensors can be configured to measure the biological product in the first and third solutions using a common measurement technique. The at least one of a measured value of a parameter of the first solution and spectral information for the first solution, and the at least one of a measured value of a parameter of the third solution and spectral information for the third solution, can be each independently selected from the group consisting of a refractive index; a conductivity; an absorbance; a transmittance; a reflectance; a concentration of the biological product; an infrared spectrum; an ultraviolet spectrum; and a Raman scattering spectrum.

The second solution may not include the biological product.

The first and second sensors can be of a different type.

The controller can be configured to determine a relative relationship between measured values of the biological product or values derived from the measured values of the biological product at the upstream and downstream locations by: obtaining a first value correlated with a concentration of the biological product or a quantity related to a concentration of the biological product in the first solution; obtaining a second value correlated with a concentration of the biological product or a quantity related to a concentration of the biological product in the third solution; calculating a comparative quantity based on the first and second values; and adjusting the at least one regulator to control the flow rate of the at least one of the first solution and the second solution based on the comparative quantity. The controller can be configured to calculate the comparative quantity as a ratio of the first and second values. The controller can be configured to calculate the comparative quantity as a mathematical function of the first and second values.

The controller can be configured to adjust the at least one regulator to control the flow rate of the at least one of the first solution and the second solution until a value of the relative relationship is within a target range of values. The controller can be configured to adjust the at least one flow regulator to control the flow rate of the at least one of the first solution and the second solution until a value of the comparative quantity is within a target range of values.

The biological product can be a protein. The protein can be an antibody, an antibody fragment, or can include a portion of an antibody.

The biological product in the first solution can be a drug substance, and the third solution can be a drug product.

The systems can include a purification unit for use in a biological manufacturing system, where the purification unit is in fluid communication with the inlet. The purification unit can include a tangential flow filtration unit.

Embodiments of the systems can also include any of the other features described herein and can include any combination of features, including combinations of features that are separately described in different embodiments, unless expressly stated otherwise.

In a further aspect, the disclosure features methods that include receiving a flowing first solution featuring a biological product and directing the flowing first solution along a flow path, where the flow path includes a dilution location at which a dilution apparatus is in fluid communication with the flow path, introducing a second solution into the flowing first solution at the dilution location to form a flowing third solution, measuring a value of an attribute of the second solution at a location upstream from the dilution location, measuring a value of an attribute of the third solution at a location downstream from the dilution location, determining a relative relationship between the measured attribute values of the second and third solutions, and adjusting a flow rate of the second solution based on the relative relationship.

Embodiments of the methods can include any one or more of the following features.

The measured value of the attribute of the second solution can be an osmolality of the second solution. The measured value of the attribute of the second solution can be a refractive index of the second solution. The measured value of the attribute of the second solution can be a conductivity of the second solution. The measured value of the attribute of the second solution can be an absorbance of the second solution. The measured value of the attribute of the second solution can be a transmittance of the second solution. The measured value of the attribute of the second solution can be a reflectance of the second solution.

The measured value of the attribute of the second solution can be measured at a single wavelength. Measuring the value of the attribute of the second solution can include obtaining a plurality of measured values of the second solution. The plurality of measured values can include spectral information for the second solution at a plurality of wavelengths.

The methods can include analyzing the spectral information to determine the value of the attribute of the second solution. Analyzing the spectral information can include using a calibrated chemometric model to determine the value of the attribute of the second solution. The value of the attribute can be an osmolality of the second solution.

The spectral information can include a Raman scattering spectrum of the second solution. The spectral information can include an infrared spectrum of the second solution. The spectral information can include an ultraviolet spectrum of the second solution.

The measured value of the attribute of the third solution can be an osmolality of the third solution. The measured value of the attribute of the second solution can be an osmolality of the second solution, and the measured value of the attribute of the third solution can be an osmolality of the third solution. The measured value of the attribute of the third solution can be at least one member of the group consisting of: a refractive index of the third solution; a conductivity of the third solution; an absorbance of the third solution; a transmittance of the third solution; and a reflectance of the third solution.

The measured value of the attribute of the third solution can be measured at a single wavelength. Measuring the value of the attribute of the third solution can include obtaining a plurality of measured values of the third solution. The plurality of measured values can include spectral information for the third solution at a plurality of wavelengths. The methods can include analyzing the spectral information to determine the value of the attribute of the third solution. Analyzing the spectral information can include using a calibrated chemometric model to determine the value of the attribute of the third solution. The value of the attribute can be an osmolality of the third solution.

The spectral information can include a Raman scattering spectrum of the third solution. The spectral information can include an infrared spectrum of the third solution. The spectral information can include an ultraviolet spectrum of the third solution.

The values of the attributes of the second and third solutions can be measured using different measurement techniques. The attributes of the second and third solutions for which values are measured can be different.

The second solution may not include the biological product. A flow rate of the flowing first solution along the flow path can be less than 2 mL/min.

Determining a relative relationship between the measured attribute values of the second and third solutions can include calculating a comparative quantity between the measured attribute values. The comparative quantity can be a ratio of the measured attribute values. The comparative quantity can be a mathematical function of the measured attribute values.

The methods can include adjusting the flow rate of the second solution until a value of the relative relationship is within a target range of values. The methods can include adjusting the flow rate of the second solution until a value of the comparative quantity is within a target range of values.

The biological product can be a protein. The protein can be an antibody, an antibody fragment, or a portion of an antibody. The biological product in the first solution can be a drug substance, and the third solution can be a drug product.

Embodiments of the methods can also include any of the other features described herein and can include any combination of features, including combinations of features that are separately described in different embodiments, unless expressly stated otherwise.

In another aspect, the disclosure features systems that include a flow channel featuring an inlet, a fluid reservoir connected to the flow channel at a dilution location, at least one flow regulator connected between the fluid reservoir and the dilution location, a first sensor positioned between the fluid reservoir and the dilution location, a second sensor positioned at a downstream location between an outlet of the flow channel and the dilution location, and a controller connected to the first and second sensors and to the flow regulator, where the flow channel is configured to receive a flowing first solution that includes a biological product through the inlet, where the fluid reservoir is configured to introduce a second solution into the flow channel at the dilution location to form a flowing third solution, where the first sensor is configured to measure an attribute value of the second solution, where the second sensor is configured to measure an attribute value of the third solution, and where the controller is configured to determine a relative relationship between the measured attribute values of the second and third solutions, and adjust the at least one flow regulator to control a flow rate of the second solution based on the relative relationship.

Embodiments of the systems can include any one or more of the following features.

The first sensor can be a Raman scattering sensor configured to measure Raman scattered light from the second solution. The first sensor can be a refractive index sensor configured to measure a refractive index of the second solution. The first sensor can be a conductivity sensor configured to measure a conductivity of the second solution. The first sensor can be an absorbance sensor configured to measure an absorbance of the second solution. The first sensor can be a transmittance sensor configured to measure a transmittance of the second solution. The first sensor can be a reflectance sensor configured to measure a reflectance of the second solution.

The first sensor can be configured to obtain a plurality of measured values of the second solution. The plurality of measured values can include spectral information for the second solution at a plurality of wavelengths. The controller can be configured to analyze the spectral information to determine the measured attribute value of the second solution. The controller can be configured to analyze the spectral information by using a calibrated chemometric model to determine the measured attribute value of the second solution from the spectral information. The first sensor can be configured to obtain a Raman scattering spectrum of the second solution.

The second sensor can be a Raman scattering sensor configured to measure Raman scattered light from the third solution. The second sensor can include at least one member of the group consisting of: a refractive index sensor configured to measure a refractive index of the third solution; a conductivity sensor configured to measure a conductivity of the third solution; an absorbance sensor configured to measure an absorbance of the third solution; a transmittance sensor configured to measure a transmittance of the third solution; and a reflectance sensor configured to measure a reflectance of the third solution.

The second sensor can be configured to obtain a plurality of measured values of the third solution. The plurality of measured values can include spectral information for the third solution at a plurality of wavelengths. The controller can be configured to analyze the spectral information to determine the measured attribute value of the third solution from the spectral information. The controller can be configured to analyze the spectral information by using a calibrated chemometric model to determine the measured attribute value of the third solution. The second sensor can be configured to obtain a Raman scattering spectrum of the third solution.

The first and second sensors can be configured to measure the attribute values of the second and third solutions using different measurement techniques. The attributes of the second and third solutions for which the values are measured can be different. The second solution may not include the biological product. The first and second sensors can be of a different type.

The controller can be configured to calculate a comparative quantity based on the measured attribute values of the second and third solutions, and adjust the at least one regulator to control the flow rate of the second solution based on the comparative quantity. The controller can be configured to calculate the comparative quantity as a ratio of the measured attribute values. The controller can be configured to calculate the comparative quantity as a mathematical function of the measured attribute values.

The controller can be configured to adjust the at least one regulator to control the flow rate of the second solution until a value of the relative relationship is within a target range of values. The controller can be configured to adjust the at least one flow regulator to control the flow rate of the second solution until a value of the comparative quantity is within a target range of values.

The biological product can be a protein. The protein can be an antibody, an antibody fragment, or a portion of an antibody. The biological product in the first solution can be a drug substance, and the third solution can be a drug product.

The systems can include a purification unit, where the purification unit is in fluid communication with the inlet. The purification unit can include a tangential flow filtration unit.

Embodiments of the systems can also include any of the other features described herein and can include any combination of features, including combinations of features that are separately described in different embodiments, unless expressly stated otherwise. As used herein, the terms “excipient” and “excipient solution” interchangeably refer to a substance, typically (although not always) in liquid form (i.e., a pure liquid, a solution consisting of one or more solvents and one or more dissolved substances, a homogeneous or heterogeneous suspension of one or more components in one or more solvents) that is generally added to a solution to form a final product solution. Typically, although not always, the solution to which the excipient is added contains one or more products from a biomanufacturing operation, and the addition of the excipient does not change the chemical nature of the one or more products in the final product solution. An excipient may, for example, contain one or more substances that help to stabilize, package, and/or deliver one or more products in the final product solution. Typically, although not always, the substances in the excipient do not react chemically with the one or more products in the solution to which the excipient is added. An excipient may be added to a product-containing solution, for example, to adjust the properties of the one or more products in the final product solution to match established specifications for the final product solution. Examples of substances which may be present in an excipient include, but are not limited to, buffers, preservatives, fillers, chelating agents, coloring agents, stabilizers/scavengers, and solvents.

As used herein, the terms “regulator” and “flow regulator” refer to any device or component of a device that can be adjusted to regulate the flow of a fluid in a conduit. Fluid valves of many different types are widely available commercially and can be used as flow regulators. Further, a wide variety of pumps are electronically controllable, and have adjustable pumping rates to regulate the flow of fluid passing through. In addition, many other devices that respond to an active control signal (e.g., an electronic signal) can function as a flow regulator. Typically, a flow regulator functions by adjusting the cross-sectional area of an aperture within the flow regulator, or adjusting a pumping rate, or both, to control the rate at which fluid passes through the flow regulator.

As used herein, a “mathematical function” that corresponds to a comparative quantity is a function of two variables, and generates an output value that is representative of the relative magnitudes of the two variables. For example, the mathematical function can be a ratio of the values of the two variables, with the value of the ratio representing the relative magnitudes of the two variables. More generally, a mathematical function that corresponds to a comparative quantity can have any functional form that generates an output value that can be used as a feedback metric to adjust the relative flow rates of fluids, as will be discussed in greater detail below. Examples of such functions include, but are not limited to, logarithmic functions, exponential functions, power law functions, polynomial functions, hyperbolic functions, and any combinations of these.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description, drawings, and claims.

### DESCRIPTION OF DRAWINGS

FIG. 1 is a schematic diagram illustrating a method by which an excipient is added in a regulated manner to a product-containing solution.

FIG. 2 is a schematic diagram illustrating another method by which an excipient is added in a regulated manner to a product-containing solution.

FIG. 3 is a schematic diagram of an example system for formulating a product in a biomanufacturing operation.

FIG. 4 is a flow chart showing a series of example steps that can be performed to implement a controlled product formulation.

FIG. 5 is a schematic diagram of another example system for formulating a product in a biomanufacturing operation.

FIG. 6 is a schematic diagram of an example of a multi-stage system for formulating a product in a biomanufacturing operation.

FIG. 7 is a graph illustrating a product formulation operation over a portion of a 30-day time period.

FIG. 8 is a graph showing temporal variations in multiple measured quantities during a product formulation process.

FIG. 9A is a graph showing measured values of protein product concentration in a final formulation as a function of time using two different excipient addition control strategies.

FIG. 9B is a graph showing measured values of osmolality for a final formulation as a function of time using two different excipient control strategies.

FIG. 10 is a schematic diagram of an example of a controller of the systems described herein.

Like reference symbols in the various drawings indicate like elements.

### DETAILED DESCRIPTION

### Introduction

Batch manufacturing operations have traditionally been used to prepare a wide variety of biological products including therapeutic agents and other drug substances. More recently, continuous biomanufacturing processes have replaced conventional batch methods, as continuous operation provides significant improvements over batch processes. For example, continuous biomanufacturing operations can generally be performed in smaller vessels than batch operations for the same product yield over time. The smaller processing volumes handled in such vessels can result in reduced mixing times and/or removal of mixing hardware such as impellers from the vessels. In turn, the absence of mechanical disturbances to solutions within the manufacturing vessels that would otherwise be caused by mixing hardware can lead to improved product quality, as vortices and mechanical shearing forces are reduced.

Continuous biomanufacturing operations can also lead to reductions in residence time within vessels for products when compared with batch operations. Product quality attributes and other attributes and manufacturing conditions can be measured in real-time or near real-time, and this information can be used to adjust conditions dynamically during manufacturing operations in a manner that would be more difficult or even incompatible with batch operations. As a result, product yields can be improved, and product lots can be generated with more predictable consistency.

Biomanufacturing operations typically involve a series of complex operations. Products are generated in a bioreactor and then extracted for further processing. Typically, post-extraction processing steps include purification in multi-column chromatography systems, buffer and salt adjustments, further polishing via multi-column chromatography, single- or multiple-stage tangential filtration, and one or more ultrafiltration and diafiltration steps.

An important aspect of continuous biomanufacturing operations involves product formulation, which generally occurs after the above stages have been performed. Product formulation generally involves adjusting the properties of a product-containing solution to match established specifications for the product solution to produce a final formulation (e.g., a formulated drug substance). For example, the product-containing solution can be adjusted so that the concentration of one or more products within the solution matches specifications for the solution in the final formulation. Alternatively, or in addition, the product-containing solution can be adjusted so that the concentration of one or more additional substances—such as, but not limited to, fillers, buffers, chelating agents, preservatives, coloring agents, stabilizers/scavengers, and other delivery agents—in the final product solution matches specifications. As a further alternative, or in addition, the product-containing solution can be adjusted so than one or more physical or chemical properties of the solution, such as (but not limited to) pH, viscosity, phase attributes, osmolality, surface tension, matches specifications.

The product formulation process typically involves adding one or more excipient solutions (“excipients”) to a product-containing solution to form a final product solution. Excipients can include substances that are intended for addition to the final product solution to adjust the concentration of substances and/or properties of the final product solution. Excipients can include one or more solvents to adjust the concentration of products in the final product solution. For example, by adding solvents volumetrically via excipient addition, the concentration of a product in the final product solution can be reduced to match established specifications for the product formulation.

In continuous biomanufacturing operations, the product formulation process occurs continuously; that is, a product-containing solution is adjusted on-the-fly to generate a final product solution. A variety of methods can be used to determine the amount of an excipient to add to a product-containing solution to produce a final product solution that is consistent with a target specification. For example, in some methods, volumetric flow rate measurements are used to regulate the addition of an excipient to a product-containing solution.

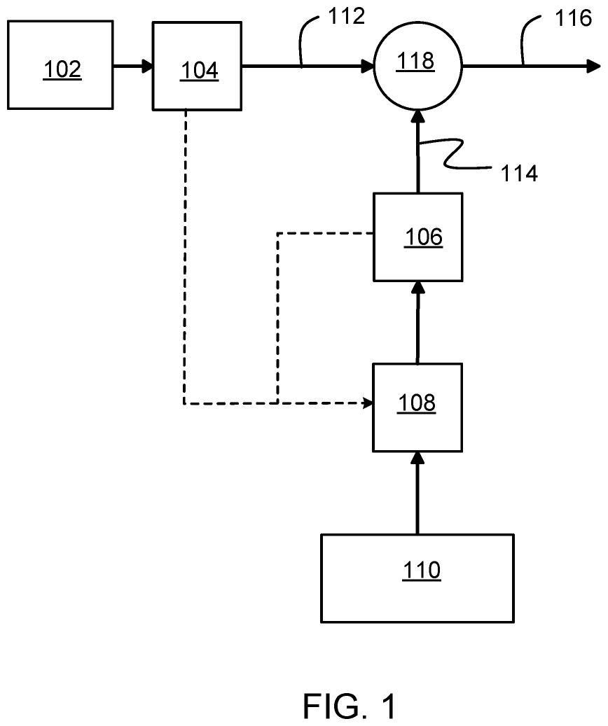

FIG. 1 is a schematic diagram illustrating a method by which an excipient is added in a regulated manner to a product-containing solution. In FIG. 1, a product-containing solution 112 is obtained from a purification unit 102 (e.g., a single-pass tangential flow filtration unit). The product-containing solution 112 flows through a flow meter 104, which measures a volumetric flow rate of solution 112. Solution 112 flows into a fluid junction 118.

An excipient 114 is stored in a reservoir 110, and transported by a pump 108 through a second flow meter 106. Flow rate measurements from flow meters 104 and 106 are used to regulate the rate at which pump 108 transports the excipient 114. The excipient 114 is transported to fluid junction 118, where it is combined with the product-containing solution 112 to form final product solution 116.

In the method illustrated in FIG. 1, the volumetric flow rate of product-containing solution 112 is used to adjust the flow rate of excipient 114 to adjust the properties of final product solution 116. For example, if the concentration of a product in solution 112 is to be reduced in final product solution 116, an appropriate amount of excipient—based on the volumetric flow rate of solution 112—is added continuously to solution 112 to achieve the desired dilution of solution 112, thereby generating a final product solution 116 in which the concentration of the product in solution 116 matches a target specification for the product concentration.

The method illustrated in FIG. 1 is effective when the flow rate of solution 112 is sufficiently large such that flow meter 104 can accurately measure the flow rate. However, for certain biomanufacturing operations, the flow rate of solution 112 (and the corresponding flow rate of excipient 114 measured by flow meter 106) may be sufficiently low so that accurate measurement of the rate becomes more difficult. For example, when purification unit 102 is a single-pass tangential flow filtration unit, the flow rate of solution 112 leaving the filtration unit can routinely be less than 2 mL/min. Under these conditions, the flow rate of excipient 114 for addition to solution 112 may be even lower, e.g., less than 0.3 mL/min. Currently available flow meters typically have relatively high measurement errors at flow rates less than about 1 mL/min. As a result, the method illustrated in FIG. 1 is prone to errors arising from both the measurement of the flow rate of solution 112, and the measurement of the flow rate of excipient 114. These errors make accurate addition of excipient 114 to solution 112 to achieve a target specification difficult and subject to unacceptable variability in some circumstances. Due to these limitations, unacceptable fluctuations in the osmolality and product concentration in the final product solution 116 have been observed experimentally.

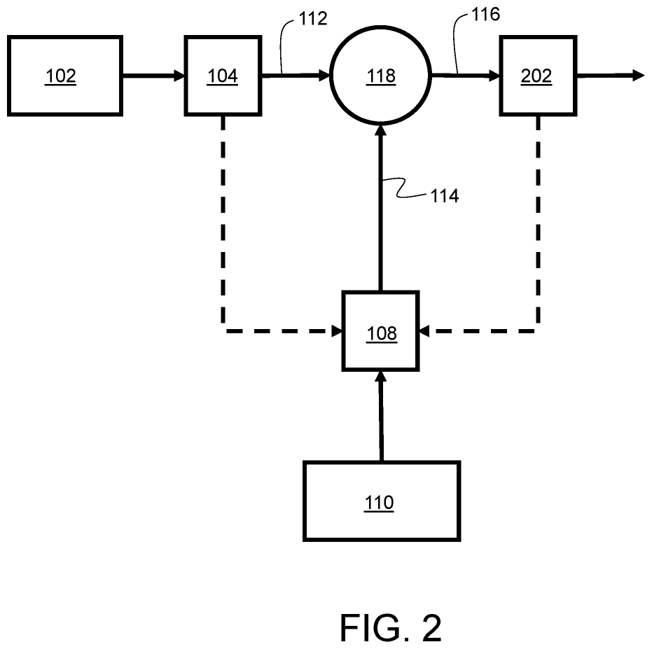

An alternative method for regulating the addition of an excipient to a product-containing solution is shown in FIG. 2. Certain components in FIG. 2 are analogous to components in FIG. 1 and are labeled with the same reference numerals. In FIG. 2, a product-containing solution 112 emerges from a purification unit 102 and the flow rate of solution 112 is measured by flow meter 104. Solution 112 passes through flow meter 104 and enters fluid junction 118.

Reservoir 110 contains excipient 114, which is pumped out of reservoir 110 and transported into fluid junction 118 by pump 108. Excipient 114 mixes with solution 112 in fluid junction 118 to produce final product solution 116, which contains the same products as solution 112. Based on the flow rate of solution 112 as measured by flow meter 104 and the assumed concentration of a product in solution 112, an appropriate flow rate of excipient 114 is determined so that following addition of the excipient to solution 112 in junction 118, the concentration of the product in solution 116 will match a target specification for solution 116.

Sensor 202 is positioned to measure a concentration of a product in solution 116 following addition of excipient 114. The measured concentration of the product in solution 116 is then used to adjust the flow rate of excipient 114 via adjustment of pump 108. In this manner, controlled adjustments to the excipient flow rate can be performed to even more closely achieve a target concentration for the product in solution 116.

However, the method illustrated in FIG. 2 is predicated on an assumption that the concentration of the product in solution 112 is relatively constant in time as solution 112 emerges from filtration unit 102. In practice, this assumption may not be valid in many circumstances. In particular, the product concentration in solution 112 may change significantly over time due to upstream changes in other processing and/or purification steps. In some circumstances, it has been experimentally observed that a single sensor 202 can be inadequate to ensure that a target specification for a product concentration in solution 116 is achieved when the product concentration in solution 112 varies substantially.

The present disclosure features methods and systems that can be used to achieve improved control over product formulation steps in continuous biomanufacturing operations. In particular, the methods and systems use a ratio-based control strategy and multiple sensor measurements to mitigate the effects of flow rates and product concentration variability on excipient addition control. By measuring the product-containing solution both pre- and post-addition of the excipient, the resulting control methodology is dynamically adaptable to a wide variety of process variations. Furthermore, many different types of sensors can be used so that robust control strategies can be implemented to ensure many different types of target specifications can be achieved for product formulations.

### Product Formulation Control Methods and Systems

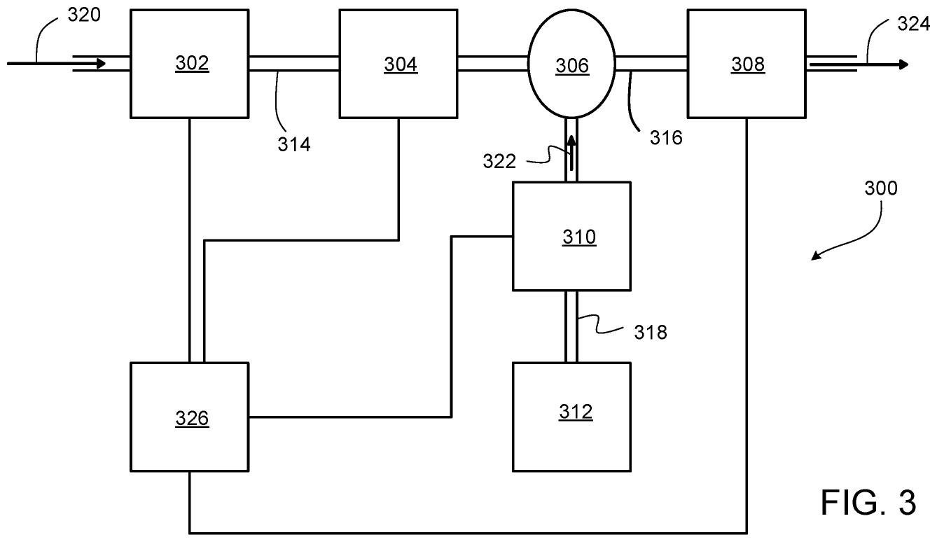

FIG. 3 is a schematic diagram showing an example of a system 300 configured to formulate a product-containing solution into a final product solution that achieves a target specification (i.e., one or more target properties) for the final product solution. System includes a flow meter 302, a first sensor 304, a fluid junction 306, a second sensor 308, a pump 310, a reservoir 312, and a controller 326. Controller 326 is connected to, and in communication with, flow meter 302, sensors 304 and 308, and pump 310. Conduits 314, 316, and 318 establish flow paths for solutions in system 300.

As shown in FIG. 3, system 300 is configured for addition of an excipient to a product-containing solution as described previously. The excipient (represented by arrow 322) is contained in reservoir 312 and transported by pump 310 to fluid junction 306. A product-containing solution (represented by arrow 320) enters system 300 from an upstream source (e.g., a filtration unit, a conduit, or another component or stage of a continuous biomanufacturing system) and mixes with excipient 322 in fluid junction 306. The resulting final product solution (represented by arrow 324) emerges into a conduit from fluid junction 306, from which it is further transported to another portion of the continuous biomanufacturing system, or for quality control verification or packaging.

System 300 includes two sensors 304 and 308 located upstream and downstream, respectively, of the location at which solution 320 is combined with excipient 322. During operation of system 300, controller 326 receives measurements from flow meter 302 and sensors 304 and 306, and determines an appropriate flow rate for excipient 322 to ensure that the attributes of final product solution 324 match target specifications for the final product solution. Controller 326 transmits control instructions to regulate pump 310, thereby controlling the rate at which excipient 322 is transported into fluid junction 306 for mixing with solution 320.

As explained above, excipient addition in a continuous biomanufacturing process generally occurs as part of a product formulation process, after products have been generated and purified. The specific nature of the excipient depends on specifications for the final product formulation. In a typical product formulation process, for example, the excipient includes a concentrated buffer that is added in sufficient quantity to match a target specification for the final product formulation. Because continuous biomanufacturing processes typically generate product-containing solutions at relatively low flow rates, addition of the excipient is also generally performed at low flow rates (for example, at 10-20% of the flow rate of the product-containing solution 320). As discussed previously, flow meters are prone to errors at the flow rates which are typical of such processes, making volumetric flow-based control methods subject to inaccuracy. Further, while the components of product-containing solutions and their concentrations can vary over time, single-measurement feedback control strategies for excipient addition frequently cannot adequately respond to such variations, yielding product formulations that are out of specification from time to time.

In contrast, the control methodology implemented by controller 326 uses both pre—and post-excipient measurements of the solution (i.e., upstream and downstream from fluid junction 306) to dynamically control the flow rate of excipient 322, and thereby ensure that the final product solution 324 matches target specifications even when properties of the product-containing solution 320—such as the concentration of a product therein—vary over time, for example, as upstream process conditions change. The product formulation process implemented by system 300 responds to such changes in automated fashion.

In particular, because sensors 304 and 308 are not flow meters, they provide measurements of solutions that are accurate even when solution flow rates are very low (or even when the solutions are not flowing at all). As such, the control methodology described herein is not prone to the errors that typically result from using flow meters at low solution flow rates. Further, because the control methodology relies on a ratio of measurements, changes in the composition of the product-containing solution are reflected in both measurements, and therefore the ratio of the measurements is insensitive to such changes.

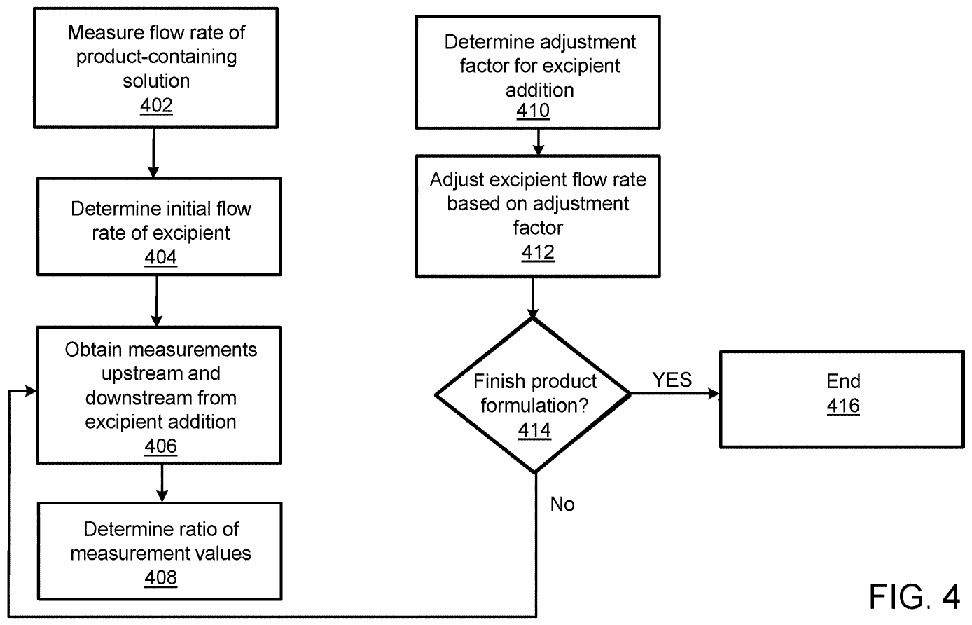

FIG. 4 is a flow chart showing an example set of steps that can be used to implement the control methodology described herein. In step 402, the flow rate of product-containing solution 320 (F) is measured by flow meter 302. Then, in step 404, controller 326 receives this measurement information and determines an initial set point for the flow rate of the excipient (Iₛₚ) as:

$${I}_{sp} = \frac{1}{( dilution factor - 1 )} \times F \qquad [ 1 ]$$

where dilution factor is a constant value that is predetermined based on the expected composition of the product-containing solution 320 and the excipient 322. Controller 326 then adjusts the flow rate of excipient 322 to this value by transmitting suitable control instructions to pump 310.

In step 406, measurement values for product-containing solution 320 (Mᵢ) and final product solution 324 (M_{f}) are obtained by sensors 304 and 308, respectively. In general, the measurement values can correspond to a wide variety of different types of measurements. In some embodiments, for example, the measurement values correspond to measurements of a concentration of a component in the product-containing solution 320 and final product solution 324, e.g., a concentration of a product in these solutions.

Controller 326 receives these measurement values, and in step 408, determines a ratio of the measurement values R as:

$$R = \frac{{M}_{f}}{{M}_{i}} \qquad [ 2 ]$$

Then, in step 410, controller 326 determines a value of an adjustment factor α for the flow rate of the excipient 322 based on the ratio of the measurement values R and a set point S that represents a target value for the ratio of the measurement values for the product-containing solution 320 and final product solution 324. As an example, if the measurement values for the two solutions represent a concentration of a component of the solutions such as a product, then the set point S represents the target value of the ratio of the concentrations of this component in the product-containing solution 320 and the final product solution 324. The adjustment factor α is then calculated as:

$$α = J ( \frac{R}{S} ) \qquad [ 3 ]$$

where J is an adjustable parameter. The value of J is typically selected such that if R>S, then the value α>1, and if R<S, then the value α<1.

Next, in step 412, controller 426 adjusts the flow rate of excipient 322 into fluid junction 306 based on the adjustment factor α. To perform this adjustment, controller 426 scales the initial set point Iₛₚ for the flow rate of the excipient 322 by the adjustment factor by calculating the product αIₛₚ. Controller 426 then transmits suitable control instructions to pump 310 to adjust the flow rate of excipient 322 to this scaled value.

In decision step 414, if product formulation is complete, then control passes to step 416 where the procedure terminates. However, if product formulation is not complete, then control returns to step 406, where further measurement values for the product-containing solution 320 and final product solution 324 are obtained by sensors 304 and 308, respectively, and a new value of the adjustment factor α and the flow rate of the excipient 322 is further adjusted based on the new value of the adjustment factor. In this manner, the control methodology dynamically responds to changes in the composition and other properties of the product-containing solution 320 as it enters system 300.

In system 300, pump 310 is used as a flow regulator to adjust the rate of flow of excipient 322 into junction 306. Pump 310 receives control signals from controller 326 which regulate the throughput of pump 310. In general, pump 310 can be implemented as any of a wide variety of adjustable pumps that are responsive to external control signals. Alternatively, or in addition, a flow regulator can be implemented in system 300 as a controllable valve that receives and responds to control signals from controller 326. For example, a constant- or variable-flow pump can be used in combination with a valve as a flow regulator in system 300 to adjust the rate at which excipient 322 introduced into junction 306. More generally, any combination of pumps, valves, and other flow-limited and/or flow-regulating components can be used to regulate the flow of excipient 322, provided that the combination provides an adjustable flow rate. In the following discussion, the term “flow regulator” is used to refer to all such combinations of components that allow fluid flow to be controlled.

In the foregoing description of system 300, controller 326 adjusts the flow rate of excipient 322 using a flow regulator. However, in some embodiments, controller 326 can adjust the flow rate of solution 320 into junction 306 as an alternative to adjusting the flow rate of excipient 322. In the control methodology described above, it is the relative flow rates of product-containing solution and excipient that are adjusted. Such an adjustment can be performed by maintaining the flow rate of either solution constant, and adjusting the other.

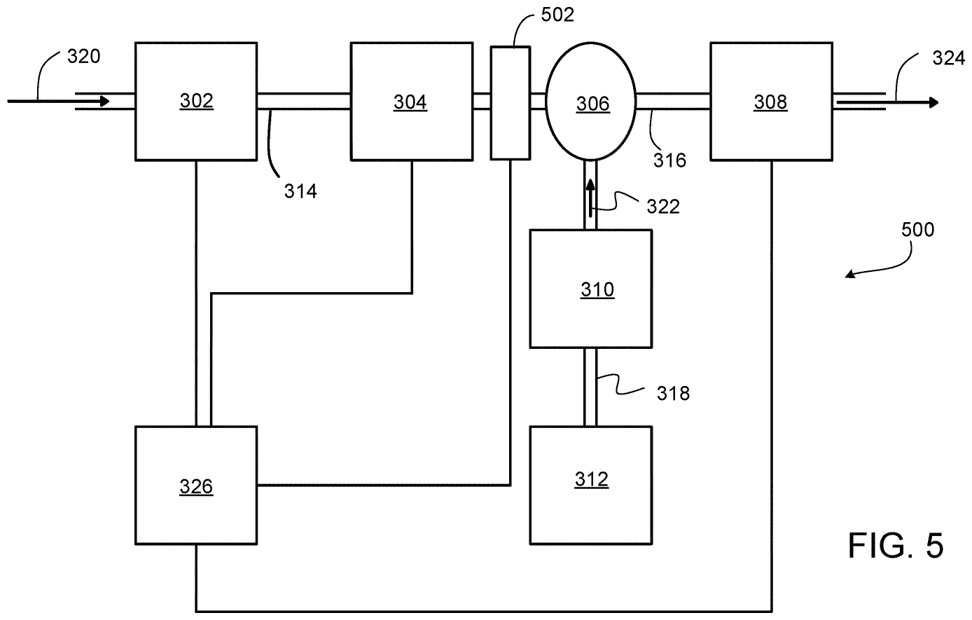

To adjust the flow of solution 320 into junction 306, a flow regulator (e.g., corresponding to any of the combinations of components discussed above) can be positioned between an inlet of the system and junction 306. FIG. 5 is a schematic diagram showing an example of a system 500 that includes a flow regulator 502 positioned to regulate the flow of solution 320 into junction 306. Flow regulator 502 is connected to controller 326 and responds to control signals from controller 326 to adjust the flow rate of solution 320. Flow regulator 502 can implemented as a valve along (e.g., with solution 320 already flowing), as a pump, as a combination of one or more pumps and one or more valves, or any other combination of components that controllably regulates the flow of solution 320.

Further, in certain embodiments, the systems described herein can actively regulate the flow rates of both solution 320 and excipient. As noted above, it is relative flow rates of these solutions that underlies the control methodology. As such, the methods described herein can be implemented by systems that include a flow regulator positioned to regulate the flow of solution 320 into junction 306 (e.g., flow regulator 502 in FIG. 5) and a flow regulator positioned to regulate the flow of excipient 322 into junction 306 (e.g., where pump 310 is positioned in FIG. 3).

As discussed above, a variety of different types of measurement values can be measured by sensors 304 and 308. In some embodiments, the measurement values correspond to measurements of a concentration or amount of a component of solutions 320 and 324 which is not present in excipient. For example, the component can be a product produced during a continuous biomanufacturing process. Such products can include, but are not limited to, proteins and fragments thereof, antibodies and fragments thereof, enzymes (e.g., therapeutic enzymes), blood factors, multispecific antibodies, nanobodies, and viral vectors.

In certain embodiments, the measurement values correspond to measurements that are related to measurements of a concentration or amount of a component of solutions 320 and 324. For example, the measurement values may correspond to a physical or chemical property of the solutions such as, but not limited to, absorbance, transmittance, reflectance, refractive index, light scattering intensity, conductivity, fluorescence intensity, and Raman scattering intensity, that are related to the presence of the component in solutions 320 and 324.

More generally, in certain embodiments, the measurement values correspond to measurements of a property of solutions 320 and 324 for which a target specification has been established in a target formulation. Such properties can include, but are not limited to, pH, viscosity, conductivity, and osmolality. Such properties can also include, but are not limited to, product quality attributes, including any of the product quality attributes described in U.S. Patent Application Publication No. US 2019/0272894, the entire contents of which are incorporated herein by reference. Chemometrics-based methods, such as those described in U.S. Patent Application Publication No. US 2019/0272894, can be used to determine values of product quality attributes from corresponding spectral information.

Sensors 304 and 308 can generally be implemented in a variety of different configurations. In certain embodiments, sensors 304 and 308 can be sensors that measure optical and/or non-optical parameters (e.g., absorbance, transmittance, reflectance, fluorescence, refractive index, Raman scattering intensity, conductivity) of solutions 320 and 324. Certain sensors are configured to measure values of these measurement quantities at a single wavelength (e.g., single-point measurements), or at multiple wavelengths (e.g., spectral or multi-point measurements). Sensors that make optical measurements at multiple wavelengths are generally referred to herein as “spectral sensors.”

Sensors that are configured to measure optical parameters can obtain measurements within a variety of wavelength regions including, but not limited to, the ultraviolet region (e.g., between 150 nm and 400 nm), the visible region (e.g., between 400 nm and 780 nm), the infrared region (e.g., between 780 nm and 3 μm).

In some embodiments, sensors 304 and 308 are implemented as the same type of sensor. More generally, however, sensors 304 and 308 do not need to be the same type of sensor, and can be implemented as different types of sensors that measure different quantities to obtain information about solutions 320 and 324. For example, sensor 304 can measure UV absorbance of solution 320, and sensor 308 can measure fluorescence of solution 324. As another example, sensor 304 can measure infrared absorbance or transmittance at multiple wavelengths, and sensor 308 can measure Raman scattering intensity at multiple wavelengths. As yet another example, sensor 304 can measure refractive index and sensor 308 can measure ultraviolet transmittance at a single wavelength. The foregoing are merely examples, and it should be understood that sensors 304 and 308 can generally be selected in any combination, measuring any of the above-described quantities, at one or more wavelengths which are the same or different, in specific embodiments of the control methodology described herein.

Sensors that are suitable for measuring each of the different parameters described above are commercially available. To measure refractive index, sensor 304 and/or sensor 308 can be implemented, for example, as a Pall mPath Reflectometer (available from Pall Corporation, New York, NY). To measure absorbance or transmittance in the ultraviolet region of the spectrum, sensor 304 and/or sensor 308 can be implemented, for example, as an Optek AF46 sensor (available from Optek International, Largo, FL), or as a PendoTECH UV photometer (available from Pendotech, Princeton, NJ). Alternatively, to measure absorbance or transmittance, sensor 304 and/or sensor 308 can be implemented as a variable path-length spectrometer such as the FlowVPE system (available from Repligen Corporation, Waltham, MA). To measure Raman scattering intensity, sensor 304 and/or sensor 308 can be implemented as the MarqMetrix system (available from MarqMetrix, Seattle, WA).

The foregoing examples of sensors are not exhaustive, and in general, the control methodology described herein can be implemented with a wide variety of different sensor types that measure many different properties of solutions 320 and 324. Preferably, sensors used to implement the control methodology obtain measurement values in real-time or near real-time (e.g., individual measurement values are obtained within a temporal window of 30 s or less).

In the embodiments described above in connection with FIGS. 3 and 4, the flow rate of the excipient 322 is adjusted according to measurement values obtained from sensors 304 and 308. More generally, however, the flow rate of solution 320 can be adjusted in addition to, or as an alternative to, the adjustment of the flow rate of excipient 322. To adjust the flow rate of solution 320, a flow regulator such as an adjustable valve can be positioned upstream of fluid junction 306, and controller 326 can transmit suitable control instructions to the flow regulator to adjust the flow rate of solution 320 into fluid junction 306. By controlling the flow rate of excipient 322, controlling the flow rate of solution 320, or controlling the flow rate of both excipient 322 and solution 320, controller 326 can adjust the properties of final product solution 324. As such, any of these different flow rate adjustments can be used in the control methodologies described herein.

In some embodiments, sensors 304 and/or 308 obtain single-point measurements of solutions 320 and/or 324. Such measurements may directly correspond to a physical or chemical property of the solutions (for example, such measurements may directly correspond to concentrations of a component in the solutions, and may be transmitted to controller 326 as concentration values). Alternatively, such measured values may be converted to measurements of a physical or chemical property of the solutions by controller 326. For example, a physical or chemical property of the solutions may be calculated by controller 326 as a mathematical function of the measured values for the solutions. The mathematical function can generally take a wide variety of forms. For example, the value of a physical or chemical property of a solution may be determined as a linear or nonlinear mathematical function of a measurement value obtained for the solution. The nonlinear function can be any of a variety of different functional types including, but not limited to, an exponential function, a logarithmic function, a polynomial function, a hyperbolic function, and a combination of any two or more functional types.

In certain embodiments, sensors 304 and/or 308 obtain multi-point measurements of solutions 320 and/or 324. Such measurements can include, for example, measurements at different times, which can be averaged, integrated, or otherwise combined to generate an output measurement that is transmitted to controller 326. Alternatively, or in addition, such measurements can include measurement values at multiple different wavelengths, i.e., spectral measurements.

Where spectral measurement values are obtained, physical or chemical properties of the solutions can be calculated as mathematical functions of measurement values at different individual wavelengths. Such mathematical functions can include multiple dependent variables corresponding to two or more of the spectral measurement values, and can be linear or nonlinear functions of each of the multiple dependent variables, as described above.

In some embodiments, where spectral measurement values are obtained by sensors 304 and/or 308, chemometrics-based methods can be used to obtain values of properties for the solutions from which the measurement values are obtained. Chemometrics-based methods can generally be applied to a wide variety of different types of measurement values, and can be used to determine values of many different types of solution properties. Chemometrics-based methods are described, for example, in U.S. Patent Application Publication No. US 2022/0101953, the entire contents of which are incorporated herein by reference.

It should also be noted that while FIGS. 3 and 4 illustrate a control methodology that can be implemented to ensure that the final product solution 324 matches target specifications for a particular parameter (e.g., a product concentration or amount, or another physical or chemical property of solution 324), multiple stages of the control methodology can be implemented in succession to ensure that an output solution matches target specifications for multiple parameters. For example, solution 324 can be directed into another system analogous to system 300, where solution 324 functions as the product-containing solution.

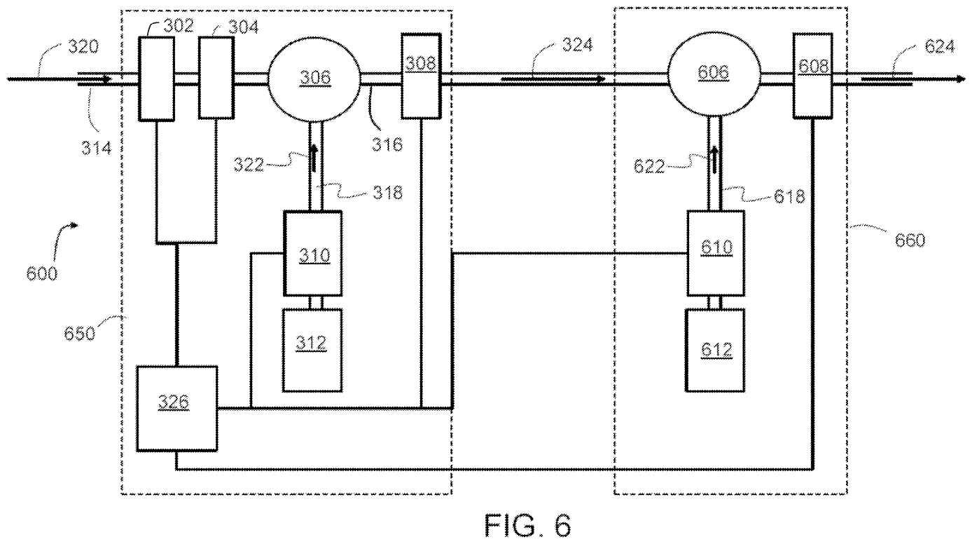

FIG. 6 is a schematic diagram of an example of a system 600 that includes two stages, each of which implements the control methodologies described herein. The first stage 650 includes components similar to the components of system 300. In general, the first stage 650 can include any of the different combinations of features, implementations, and embodiments described above.

Solution 324 emerges from the first stage 650 and enters the second stage 660 which includes a fluid junction 606, a third sensor 608, a pump 610, and a fluid reservoir 612 that contains a second excipient 622. Pump 610 and fluid reservoir 612 are connected to fluid junction by conduit 618.

During operation of system 600, first stage 650 functions as described above to produce a solution 324 which matches a target specification for a particular property or constituent (e.g., a first product present in solution 324). Solution 324 enters second stage 660 which also functions as described above, but introduces second excipient 622 into solution 324. Sensor 608 measures the solution after addition of excipient 622, and the measured value(s) are transmitted to controller 326. Controller 326 adjusts the relative rate at which excipient 622 and solution 324 enter fluid junction 606 to ensure that solution 624 matches another target specification, i.e., different from the target specification for first stage 650. As an example, first stage 650 and second stage 660 can operate to ensure that the concentration for two different products present initially in solution 320 match target specifications in solution 624. As another example, first stage 650 and second stage 660 can operate to ensure that the concentration for a product and another chemical or physical property of solution 624 match target specifications.

In general, both first stage 650 and second stage 660 can include any of the components, implementations, and embodiments described herein, including any of the flow regulators and sensors (and different combinations thereof) that are described. The two stages can adjust any combination of different component and properties of incoming solution 320. Further, in certain embodiments, some components described herein may be omitted. For example, in system 600, sensor 608 functions as the downstream sensor in second stage 660, while sensor 308 functions as the downstream sensor in first stage 650 and as the upstream sensor in second stage 660. Alternatively, in some embodiments, second stage 660 can include another sensor positioned between sensor 308 and fluid junction 606 that is connected to controller 326 and functions as the upstream sensor in second stage 660. This additional sensor can be any of the different types of sensors described herein.

The systems described herein are not limited to only two stages as shown in FIG. 6. In general, the systems can include any number (e.g., two or more, three or more, four or more, five or more, six or more, eight or more, ten or more, or even more) stages arranged sequentially. By implementing sequential stages analogous to system 600, compliance with multiple target specifications can be achieved.

### Hardware and Software Implementations

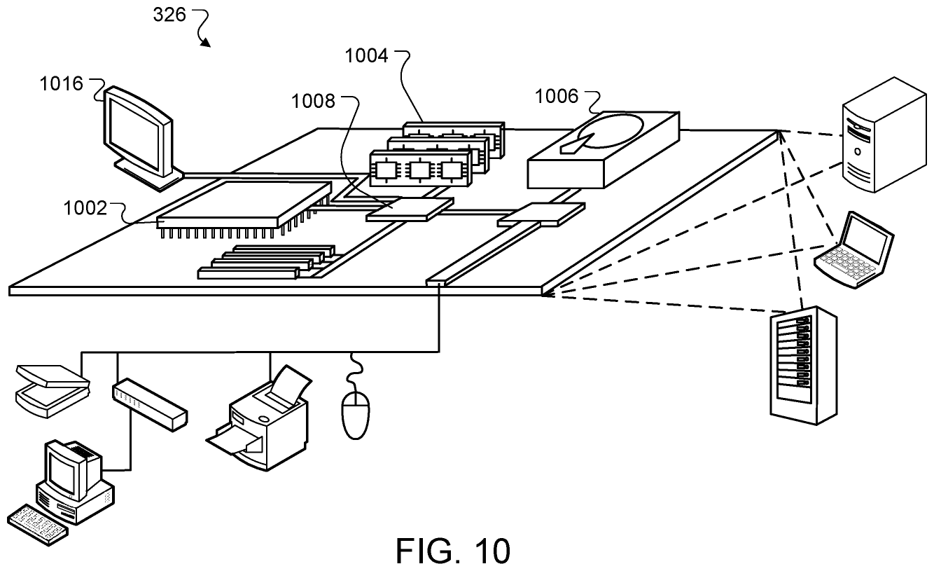

FIG. 10 shows an example of controller 326, which may be used with the systems and methods disclosed herein. Controller 326 can include one or more processors 1002, memory 1004, a storage device 1006 and interfaces 1008 for interconnection. The processor(s) 1002 can process instructions for execution within the controller, including instructions stored in the memory 1004 or on the storage device 1006. For example, the instructions can instruct the processor 1002 to perform any of the analysis and control steps disclosed herein.

The memory 1004 can store executable instructions for processor 1002, information about parameters of the system such as excitation and detection wavelengths, and measured image information. The storage device 1006 can be a computer-readable medium, such as a floppy disk device, a hard disk device, an optical disk device, or a tape device, a flash memory or other similar solid state memory device, or an array of devices, including devices in a storage area network or other configurations. The storage device 1006 can store instructions that can be executed by processor 1002 as described above, and any of the other information that can be stored by memory 1004.

In some embodiments, controller 326 can include a graphics processing unit to display graphical information (e.g., using a GUI or text interface) on an external input/output device, such as display 1016. The graphical information can be displayed by a display device (e.g., a CRT (cathode ray tube) or LCD (liquid crystal display) monitor) for displaying any of the information, such as measured and calculated spectra and images, disclosed herein. A user can use input devices (e.g., keyboard, pointing device, touch screen, speech recognition device) to provide input to controller 326. In some embodiments, one or more such devices can be part of controller 326.

A user of any of the systems described herein can provide a variety of different types of instructions and information to controller 326 via input devices. The instructions and information can include, for example, target specifications for any of the formulated substances produced via a biomanufacturing operation and system, information about excipients, information and selections of measured values, and calibration information for any of the components of the systems and method steps described herein. Controller 326 can use any of these various types of information to perform the methods and functions described herein. It should also be noted that any of these types of information can be stored (e.g., in storage device 1006) and recalled when needed by controller 326.

The methods and individual steps disclosed herein can be implemented by controller 326 by executing instructions in one or more computer programs that are executable and/or interpretable by the controller 326. These computer programs (also known as programs, software, software applications or code) include machine instructions for a programmable processor, and can be implemented in a high-level procedural and/or object-oriented programming language, and/or in assembly/machine language. For example, computer programs can contain the instructions that can be stored in memory 1004, in storage unit 1006, and/or on a tangible, computer-readable medium, and executed by processor 1002 as described above. As used herein, the term “computer-readable medium” refers to any computer program product, apparatus and/or device (e.g., magnetic discs, optical disks, memory, Programmable Logic Devices (PLDs), ASICs, and electronic circuitry) used to provide machine instructions and/or data to a programmable processor, including a machine-readable medium that receives machine instructions.

By executing instructions as described above (which can optionally be part of controller 326), the controller can be configured to implement any one or more of the various steps described in connection with any of the control methodologies described herein. For example, controller 326 can receive measured values from sensors and flow meters, calculate any of the quantities described herein (and other quantities as well), determine actions based on quantities and other decision criteria, and transmit control instructions to the components of any of the systems described herein.

### Applications

Although the control methodologies described herein are particularly applicable to formulation processes in continuous biomanufacturing operations, they can also be applied to product formulation processes in other biomanufacturing operations as well such as batch operations. Further, while the control methodologies are described herein in the context of product formulation processes, the methodologies can also be applied to additional upstream steps (e.g., prior to product formulation) in biomanufacturing operations. For example, the methodologies can be applied to additions of salts, solvents, detergents, and other agents, and dilutions of process fluids, and more generally, to any operation or step in which a target specification for an output solution or fluid stream has been established.

For example, in some embodiments, the control methodologies described herein can be applied to an intermediate process (e.g., a process implemented following extraction of a product from a bioreactor, but prior to product formulation) in which a process fluid is diluted to reduce a concentration of the components in the fluid. Any of the various types of measured values described above can be used to regulate the relative flow of fluids to perform the dilution. The measured values may correlate with the product in the fluid, or they may correct with another non-product species in the fluid, as all components of the fluid are similarly diluted by the addition of another fluid. The fluid that is added for purposes of dilution may contain any of the components described herein in connection with excipients, and in addition to diluting the process fluid, may add to the process fluid additional components. For example, the solution that is added may contain one or more salts so that the process fluid is simultaneously diluted and it salt content is increased using the methods and systems described herein. As another example, the fluid that is added to the process fluid may contain a detergent that is added to the process fluid concomitant with dilution.

The foregoing dilution operations can be implemented at multiple different stages of a biomanufacturing operation. For example, dilution can be used as a load adjustment step during purification operations to ensure that chromatography systems are adequately loaded but not overloaded. Dilution operations can also be implemented as a precursor to other unit operations such as polishing and filtration steps.

### Examples

### Example 1: Continuous Biomanufacturing

The control methodology described above was implemented over a 30-day period in a continuous biomanufacturing operation performed in a 500 L bioreactor. The operation generated process fluids containing a protein product, and protein concentration in the final product solution was adjusted to match a target specification.

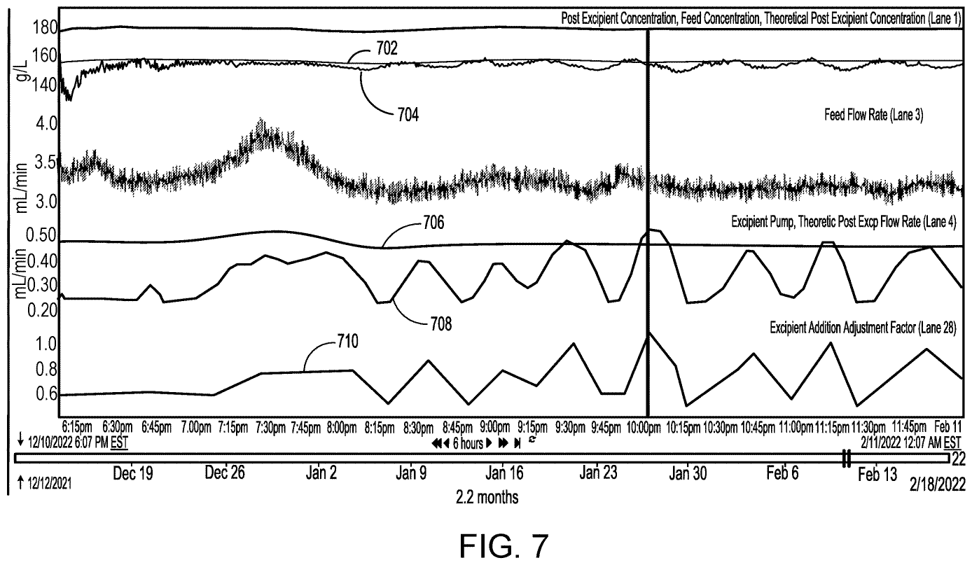

FIG. 7 is a graph illustrating performance of the control methodology over a portion of the 30-day time period. In FIG. 7, trace 702 corresponds to the target concentration of the protein in the final product solution, and trace 704 corresponds to the measured protein concentration in the solution. As shown in FIG. 7, the measured protein concentration approximately matches the target protein concentration until the time corresponding to the vertical line, at which time the measured concentration of the protein is less than the target concentration. If the flow rate of the excipient (trace 708) was maintained at the rate determined from volumetric flow measurements (e.g., determined solely based on the flow rate measured by flow meter 302), then the excipient flow rate represented by trace 706 would be consistently too large, and the protein concentration in the final product solution would be consistently too low.

However, by implementing the control methodology described herein, the measured protein concentration was successfully adjusted. In FIG. 7, at the time indicated by the vertical line, the adjustment factor α (trace 710) calculated by controller 326 decreased, and controller 326 therefore lowered the flow rate of the excipient added to the product-containing solution. As a result, the measured concentration 704 of the protein in the final product solution increased to match the target protein concentration 702 within 30 minutes of the initial deviation of the protein concentration from the target concentration.

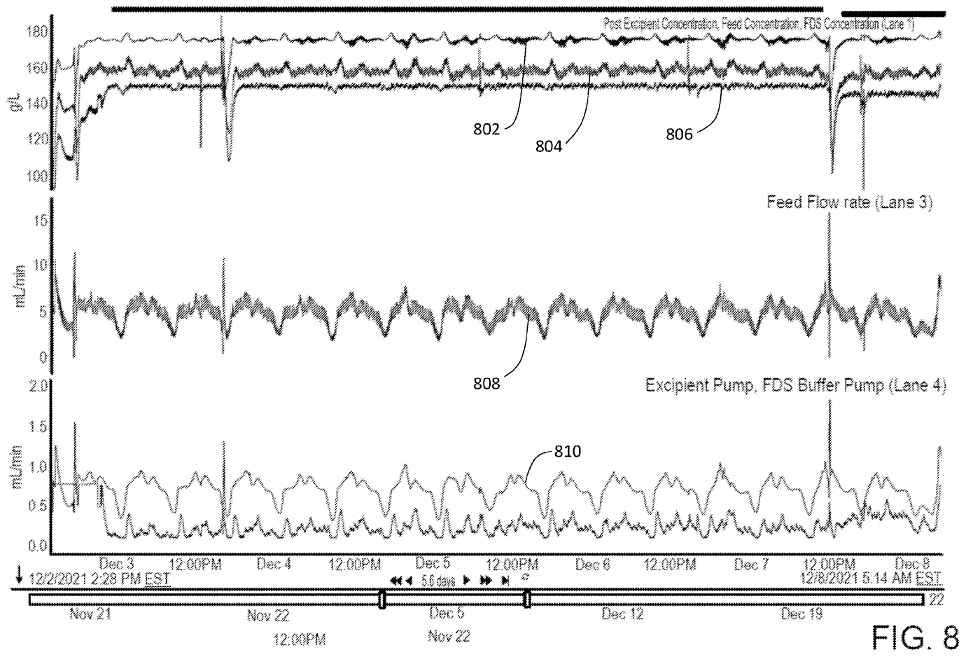

FIG. 8 is a graph showing temporal variations in multiple measured quantities during a product formulation process implemented with the control methodology described herein. In FIG. 8, trace 802 shows the measured protein concentration in the solution prior to addition of the excipient (e.g., measured by sensor 304), trace 804 shows the measured protein concentration following addition of the excipient (e.g., measured by sensor 308), and trace 806 shows the measured protein concentration in the product formulation. Trace 808 shows the measured flow rate of the product-containing solution (e.g., measured by flow meter 302), and trace 810 shows the flow rate of the excipient that was added to the product-containing solution. As is evident from the figure, each of these quantities fluctuated measurably over time. Nonetheless, as indicated by trace 806 which corresponds to concentration of the protein in the product formulation, the control methodology was able to compensate for these temporal fluctuations, yielding a final formulation in which the protein concentration remained approximately constant over the time window shown in the figure.

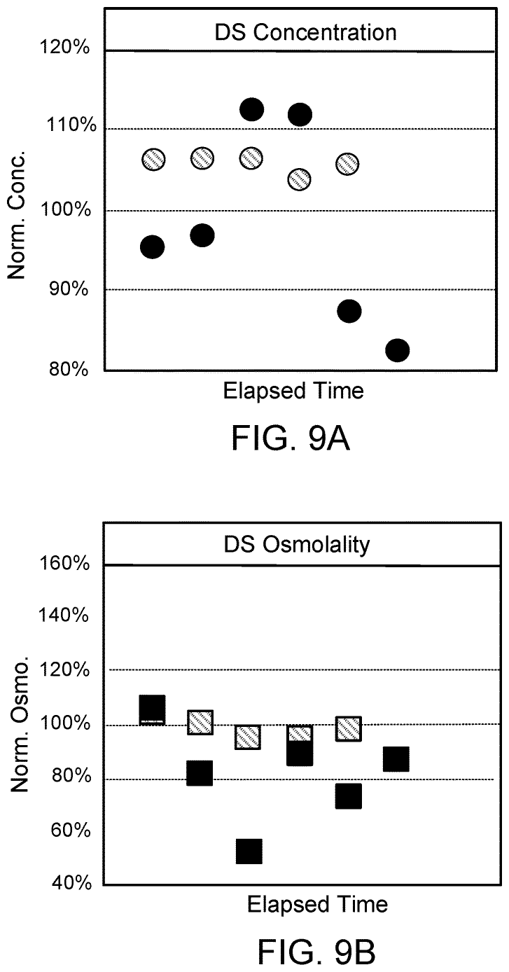

FIG. 9A is a graph showing measured values of protein product concentration in the final product solution as a function of time using a control strategy based on only measured volumetric flow rate of the product-containing solution (solid circles), as described above in connection with FIG. 1, and using the control methodology described in connection with FIGS. 3 and 4 (hatched circles). FIG. 9B is a graph showing measured values of osmolality for the final product solution as a function of time using a control strategy based on only measured volumetric flow rate of the product-containing solution (solid circles), as described above in connection with FIG. 1, and using the control methodology described in connection with FIGS. 3 and 4 (hatched circles). From the data shown in these figures, it is evident that by using the two-sensor control methodology described herein, fluctuations in both protein concentration and osmolality of the final product solution were substantially reduced, with a variation of less than 5% over the time window represented in the figures, relative to fluctuations of more than 20% in the measured values of protein concentration and osmolality that were observed when using a control strategy based on only measured volumetric flow rate of the product-containing solution.

### Example 2: Osmolality-Based Control

In this example, measurements of osmolality were used to control excipient addition during a biomanufacturing process. Osmolality is a typical critical quality attribute during formulation as it provides a confirmation of solute content and can be used to detect deviations in compounding. As used herein, osmolality is defined as the total concentration of all osmotically active dissolved solutes present in a given volume of solution. Osmolality can be measured by an osmometer which determines the freezing point of an aqueous solution with one or more dissolved solutes. Because dissolved solutes depress the freezing point of a solution relative to the corresponding pure solvent, osmolality can be determined from a measurement of a solution's freezing point.

Conventional measurement of osmolality occurs off-line, since the solution is frozen during the process. Thus, to implement an osmolality-based control strategy in continuous biomanufacturing, a sensor capable of measuring osmolality at higher measurement frequency (for example, in real time or near real time, without freezing the solution) is desirable.

Raman spectroscopy has been widely used in bioprocessing and has been previously used in both upstream and downstream applications. Raman spectroscopy provides measurement of inelastic light scattering by a sample that is typically excited with light (e.g., laser light) in the visible, near infrared, or near ultraviolet range. Raman scattered light is typically energy-shifted relative to the excitation light, and the energy shift provides insight into vibrational modes of the sample. By resolving the frequencies of the Raman scattered light (e.g., by directing the scattered light to pass through a monochromator and spatially separating the frequencies of the scattered light for detection by a detector), a variety of sample attributes—including, but not limited to, the identity and/or concentration of various components of the sample—can be determined.

Raman scattering measurements can be performed rapidly, and are therefore well-suited for implementation in any of the methods described herein. As described previously, multiple process parameters can be extracted from Raman spectra using univariate or multivariate analysis techniques. These process parameters can be used for automatic recipe progression and/or closed-loop control in any of the foregoing methods.

In this example, a Raman scattering sensor was used to obtain measurements of Raman scattering intensity, and quantify osmolality of a product-containing solution over a pre-defined range of target osmolality in a continuous formulation excipient addition step. To evaluate the effectiveness of using Raman scattering for osmolality-based control, each expected formulation component for a product-containing solution (e.g., mAb protein, excipient 1, excipient 2) was evaluated for its Raman activity at concentrations expected to be observed during normal processing. Test solutions of each formulation component were prepared in water at several concentration levels, along with test solutions of the excipient buffer and the target formulated drug substance. Each of these target solutions were then measured with a Raman spectrometer (obtained from MarqMetrix, Seattle, WA) with a 180 uL flow cell. The spectrometer's laser power was 450 mW and the exposure time was optimized to achieve a detector saturation level between 50 to 80%. The number of scans was chosen to reach a sufficient spectral quality and desired measurement time. In consideration of the control methods described herein, the various measurement conditions were selected to achieve a measurement time of 20 seconds or less.

The acquired Raman spectra were overlaid to identify which formulation components were visible within the measurement wavelength range. Formulated product (e.g., drug substance) target spectra were composed based on the identified Raman-visible components. It was observed that the Raman scattering methods were capable of measuring the following types of formulation components: stabilizing salts and tonicity modifiers (i.e., arginine); sugars; cryoprotectants; and lyoexcipients (i.e., sucrose).

Other formulation components were less visible in the measured Raman spectra. These included surfactants (i.e., PS80) and chelators (i.e., EDTA). Without being bound by theory, it is possible that these components were less visible due to their relatively low target concentrations in the product-containing solution.

To develop a Raman model for osmolality measurement to enable monitoring and automated feed-back control as described herein, a calibration dataset of Raman spectra and off-line reference measurements was obtained by creating a synthetic flow path that mimicked the unit operation conditions. The flow path began with a solution of buffer and protein at high concentration and over time a solution of the excipient buffer was added in increasing steps. While the flow path was running, Raman spectra from 25 to 3500 cm were obtained under conditions similar to those described above. Off-line samples were also collected and tested for osmolality by conventional measurements using an osmometer.

Models were developed using the multivariate modelling software SIMCA® by first trimming the spectra to spectral regions previously identified during measurements of the test solutions. After spectral trimming, pre-processing using first derivative, Savitzky-Golay smoothing, and standard normal variate (SNV) normalization was applied. Pre-processed spectra were matched to the off-line reference measurements and PLS models for osmolality were created. Latent variables for each model were chosen based on leave-one-out cross validation and selecting the minimum root mean squared error of calibration and cross-validation. In general, the model selected for use had a low root mean squared error of calibration and cross-validation and had R² and Q² values greater than 90%, indicating the ability to accurately estimate new data. Moreover, the selected model covered a range of osmolality values that were expected to be encountered during continuous formulation operations, helping ensure that the model would not have to predict osmolality values by extrapolating outside of the calibration range (e.g., osmolality values ranged from 60 to 700 mOsm/kg H₂O, with the target osmolality being 383 mOsm/kg H₂O).

To perform process control for continuous formulation of a product, Raman spectra were obtained by using the MarqMetrix Raman spectrometer as a sensor. The spectrometer included a 180 uL flow cell connected to MarqMetrix Raman suite. The Raman spectral information obtained by the sensor was sent to synTQ, a process analytical technology data management tool, using the OPC communication protocol. This tool used SIMCA® QP to convert the Raman spectral information into an osmolality value. This value was transmitted by the synTQ tool to control hardware for the system and entered into a respective proportional integral derivative (PID) controller-based loop for process control.

In this example, Raman measurements were obtained from a product-containing solution immediately after excipient addition to continuously measure the osmolality and product concentration. When Raman-determined osmolality was used for control in this step, a setpoint value for target osmolality was input into the controller, and a feedback loop using PID-based control was used to adjust the flow rate of the excipient pump. During the run, the performance of the model was assessed by periodically removing and analyzing off-line samples and comparing the in-line and off-line measurements. Model performance was assessed by calculating the root mean square error of prediction (RMSEP). Raman-based osmolality predictions were considered acceptable if the RMSEP was below 5 mOsm/kg H₂O. Nonetheless, based on typical historical process variation during formulation, a RMSEP up to 20 mOsm/kg H₂O was also considered acceptable.

In many embodiments, the process control methods described herein involve determining a ratio of measurements of a particular attribute value, and adjusting relative flow rates of excipient and/or product solutions based on the measurement ratio. Osmolality ratio-based control, as described previously, was also tested in this example.

Typically, when excipient is added to a product-containing solution as part of a product formulation, the excipient buffer is concentrated at a specific proportion to the final product stream. As this ratio of excipient is fixed, the ratio of the osmolality of the excipient buffer to the post-excipient product stream will also be at the same value. By determining the osmolality of the excipient buffer and post-excipient product stream continuously from Raman measurements using chemometrics-based models for both solutions, the ratio of the osmolality values for the excipient buffer and post-excipient product stream was used to adjust the flow rate of the excipient pump.

Similar to the methods described herein for control based on attribute value ratios such as a protein concentration ratio, the buffer:product adjustment factor was continuously calculated to adjust the excipient pump flow rate for process control. If the adjustment factor<1, the excipient pump flow rate was decreased to bring the product stream closer to the target osmolality value, while the inverse is true for an adjustment factor>1.

It should be noted that in some embodiments, if there is significant contribution from protein to the osmolality value of the post-excipient product-containing solution, either a baseline value for protein osmolality can be subtracted from the post-excipient osmolality determined as described above, or a third measurement of osmolality can be obtained for the pre-excipient product-containing solution, and this osmolality value can be continuously subtracted based on the performance of the previous unit operation. Osmolality values for the pre-excipient product-containing solution can generally be obtained using any of the methods described herein, including by performing Raman scattering measurements and applying multivariate models to obtained Raman spectral information to extract osmolality values.

Raman scattering measurements can generally be used to obtain osmolality values for any of the solutions in a continuous biomanufacturing system. In this example, Raman scattering measurements are used to measure osmolality of post-excipient, pre-excipient, and excipient buffer solutions. However, it should be understood that Raman scattering measurements can also be used to obtain osmolality measurements for other process solutions including, but not limited to, process solutions arising after one or more filtration, dilution, blending, separation, and/or polishing steps. Such solutions can include, for example, filtrate solutions, eluted solutions from chromatography columns, solutions obtained from mixing vessels, solutions obtained from centrifuges, and solutions obtained from process sampling at many different locations in a biomanufacturing system.

As noted above, osmolality is a typical critical quality attribute during formulation and can be used to detect deviations in compounding. In this example, Raman scattering measurements were used to measure osmolality. However, other types of measurements and corresponding sensors can also be used to obtain osmolality measurements for use in the methods described herein. For example, infrared and/or ultraviolet absorbance or reflectance measurements can be used to obtain spectra for a solution, and multivariate chemometrics-based methods can be applied to the spectra to obtain osmolality values. As another example, conductivity sensors can be used to measure the conductivity of solutions, and osmolality values for the solutions can be determined from the measured conductivity.

In this example, process control was performed by measuring the osmolality of the excipient and the post-excipient solutions for ratio-based adjustment of the excipient flow rate. It should be noted that while osmolality is a useful attribute for feedback control, measurements of any of the other attributes described herein for the excipient and the post-excipient solutions can also be used for ratio-based adjustment of the excipient flow rate in the same manner. That is, adjustment of the excipient flow rate based on a ratio of attribute values for the excipient and post-excipient solutions is not restricted to only the osmolality attribute, but can also be performed based on measurements of a wide variety of different attribute values for the solutions including, but not limited to, any of the attributes described generally herein.

### OTHER EMBODIMENTS

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method, comprising:
receiving a flowing first solution comprising a biological product and directing the flowing first solution along a flow path, wherein the flow path comprises a dilution location at which a dilution apparatus is in fluid communication with the flow path;
introducing a second solution into the flowing first solution at the dilution location to form a flowing third solution;
measuring the biological product in the flowing first solution at a location upstream from the dilution location;
measuring the biological product in the flowing third solution at a location downstream from the dilution location;
determining a relative relationship between measured values of the biological product or values derived from measured values of the biological product at the upstream and downstream locations; and
adjusting a flow rate of at least one of the first solution and the second solution based on the relative relationship.

2. The method of claim 1, wherein measuring the biological product at the upstream location comprises obtaining a measured value of a parameter of the first solution.

3. The method of claim 2, wherein the measured value of the parameter is a refractive index of the first solution.

4. The method of claim 2, wherein the measured value of the parameter is a conductivity of the first solution.

5. The method of claim 2, wherein the measured value of the parameter is an absorbance of the first solution.

6. The method of claim 2, wherein the measured value of the parameter is a transmittance of the first solution.

7. The method of claim 2, wherein the measured value of the parameter is a reflectance of the first solution.

8. The method of claim 2, wherein the measured value of the parameter is a concentration of the biological product in the first solution.

9. The method of claim 2, wherein the measured value of the parameter is measured at a single wavelength.

10. The method of claim 9, wherein the single wavelength is in an ultraviolet spectral region.

11. The method of claim 9, wherein the single wavelength is in a visible spectral region.

12. The method of claim 9, wherein the single wavelength is in an infrared spectral region.

13. The method of claim 1, wherein measuring the biological product at the upstream location comprises obtaining a plurality of measured values of the first solution.

14. The method of claim 13, wherein the plurality of measured values comprise spectral information for the first solution at a plurality of wavelengths.

15. The method of claim 14, further comprising analyzing the spectral information to determine a value derived from the spectral information.

16. The method of claim 15, wherein analyzing the spectral information comprises using a calibrated chemometric model to determine the value derived from the spectral information.

17. The method of claim 15, wherein the value derived from the spectral information is a concentration of the biological product in the first solution, or a quantity related to a concentration of the biological product in the first solution.

18. The method of claim 14, wherein the spectral information comprises an infrared spectrum of the first solution.

19. The method of claim 14, wherein the spectral information comprises an ultraviolet spectrum of the first solution.

20. The method of claim 14, wherein the spectral information comprises a Raman scattering spectrum of the first solution.

21. The method of claim 18, further comprising measuring attenuated total reflection of incident infrared light to obtain the infrared spectrum.

22. The method of claim 3, further comprising measuring the refractive index of the first solution by measuring attenuated total reflection of incident infrared light from the first solution.

23. The method of claim 1, wherein measuring the biological product at the downstream location comprises obtaining a measured value of a parameter of the third solution.

24. The method of claim 23, wherein the measured value of the parameter of the third solution comprises at least one member of the group consisting of: a refractive index of the third solution; a conductivity of the third solution; an absorbance of the third solution; a transmittance of the third solution; a reflectance of the third solution; and a concentration of the biological product in the third solution.

25. The method of claim 23, wherein the measured value of the parameter of the third solution is measured at a single wavelength.

26. The method of claim 25, wherein the single wavelength comprises a wavelength in an ultraviolet, visible, or infrared spectral region.

27. The method of claim 1, wherein measuring the biological product at the downstream location comprises obtaining a plurality of measured values of the third solution.

28. The method of claim 27, wherein the plurality of measured values comprise spectral information for the third solution at a plurality of wavelengths.

29. The method of claim 28, further comprising analyzing the spectral information to determine a value derived from the spectral information.

30. The method of claim 29, wherein analyzing the spectral information comprises using a calibrated chemometric model to determine the value derived from the spectral information.

31. The method of claim 29, wherein the value derived from the spectral information is a concentration of the biological product in the third solution, or a quantity related to a concentration of the biological product in the third solution.

32. The method of claim 28, wherein the spectral information comprises at least one member of the group consisting of an infrared spectrum of the third solution, an ultraviolet spectrum of the third solution, and a Raman scattering spectrum of the third solution.

33. The method of claim 32, further comprising measuring attenuated total reflection of incident infrared light to obtain the infrared spectrum.

34. The method of claim 24, further comprising measuring the refractive index of the third solution by measuring attenuated total reflection of incident infrared light from the third solution.

35. The method of claim 1, wherein:
measuring the biological product at the upstream location comprises obtaining at least one of a measured value of a parameter of the first solution and spectral information for the first solution;
measuring the biological product at the downstream location comprises obtaining at least one of a measured value of a parameter of the third solution and spectral information for the third solution; and
the at least one of a measured value of a parameter of the first solution and spectral information for the first solution is measured using a different measurement technique from the at least one of a measured value of a parameter of the third solution and spectral information for the third solution.

36. The method of claim 35, wherein the at least one of a measured value of a parameter of the first solution and spectral information for the first solution comprises a different type of information from the at least one of a measured value of a parameter of the third solution and spectral information for the third solution.

37. The method of claim 35, wherein the at least one of a measured value of a parameter of the first solution and spectral information for the first solution, and the at least one of a measured value of a parameter of the third solution and spectral information for the third solution, are each independently selected from the group consisting of a refractive index; a conductivity; an absorbance; a transmittance; a reflectance; a concentration of the biological product; an infrared spectrum; an ultraviolet spectrum; and a Raman scattering spectrum.

38. The method of claim 1, wherein:
measuring the biological product at the upstream location comprises obtaining at least one of a measured value of a parameter of the first solution and spectral information for the first solution;
measuring the biological product at the downstream location comprises obtaining at least one of a measured value of a parameter of the third solution and spectral information for the third solution; and
the at least one of a measured value of a parameter of the first solution and spectral information for the first solution and the at least one of a measured value of a parameter of the third solution and spectral information for the third solution are measured using a common measurement technique.

39. The method of claim 38, wherein the at least one of a measured value of a parameter of the first solution and spectral information for the first solution, and the at least one of a measured value of a parameter of the third solution and spectral information for the third solution, are each independently selected from the group consisting of a refractive index; a conductivity; an absorbance; a transmittance; a reflectance; a concentration of the biological product; an infrared spectrum; an ultraviolet spectrum; and a Raman scattering spectrum.

40. The method of claim 1, wherein the second solution does not comprise the biological product.

41. The method of claim 1, wherein a flow rate of the flowing first solution along the flow path is less than 2 mL/min.

42. The method of claim 41, wherein the flow rate is less than 1 mL/min.

43. The method of claim 1, wherein determining a relative relationship between measured values of the biological product or values derived from the measured values of the biological product at the upstream and downstream locations comprises:
obtaining a first value correlated with a concentration of the biological product or a quantity related to a concentration of the biological product in the first solution at the upstream location;
obtaining a second value correlated with a concentration of the biological product or a quantity related to a concentration of the biological product in the third solution at the downstream location;
calculating a comparative quantity based on the first and second values; and
adjusting the flow rate of the at least one of the first solution and the second solution based on the comparative quantity.

44. The method of claim 43, wherein the comparative quantity is a ratio of the first and second values.

45. The method of claim 43, wherein the comparative quantity is a mathematical function of the first and second values.

46. The method of claim 1, further comprising adjusting the flow rate of the at least one of the first solution and the second solution until a value of the relative relationship is within a target range of values.

47. The method of claim 43, further comprising adjusting the flow rate of the at least one of the first solution and the second solution until a value of the comparative quantity is within a target range of values.

48. The method of claim 1, wherein the biological product is a protein.

49. The method of claim 48, wherein the protein is an antibody, an antibody fragment, or comprises a portion of an antibody.

50. The method of claim 1, wherein the biological product in the first solution is a drug substance, and wherein the third solution is a drug product.

51. The method of claim 1, wherein the biological product is a first biological product, the dilution location is a first dilution location, and wherein the flow path comprises a second dilution location downstream from the first dilution location, the method further comprising:
introducing a fourth solution into the flowing third solution at the second dilution location to form a flowing fifth solution, wherein the second dilution location is downstream from a location at which the first biological product is measured in the flowing third solution;
measuring a second biological product in the flowing fifth solution at locations upstream and downstream from the second dilution location;
determining a relative relationship between measured values of the second biological product or values derived from the measured values of the second biological product at the upstream and downstream locations; and
adjusting at least one of a flow rate of the third solution and a flow rate of the fourth solution based on the relative relationship for the second biological product.

52. The method of claim 51, wherein measuring the second biological product at the location downstream from the second dilution location comprises obtaining a measured value of a parameter of the fifth solution.

53. The method of claim 52, wherein the measured value of the parameter of the fifth solution comprises at least one member of the group consisting of: a refractive index of the fifth solution; a conductivity of the fifth solution; an absorbance of the fifth solution; a transmittance of the fifth solution; a reflectance of the fifth solution; and a concentration of the second biological product in the fifth solution.

54. The method of claim 52, wherein the measured value of the parameter of the fifth solution is measured at a single wavelength.

55. The method of claim 54, wherein the single wavelength comprises a wavelength in an ultraviolet, visible, or infrared spectral region.

56. The method of claim 51, wherein measuring the second biological product at the location downstream from the second dilution location comprises obtaining a plurality of measured values of the fifth solution.

57. The method of claim 56, wherein the plurality of measured values comprise spectral information for the fifth solution at a plurality of wavelengths.

58. The method of claim 57, further comprising analyzing the spectral information for the fifth solution to determine a value derived from the spectral information.

59. The method of claim 58, wherein analyzing the spectral information for the fifth solution comprises using a calibrated chemometric model to determine the value derived from the spectral information.

60. The method of claim 58, wherein the value derived from the spectral information is a concentration of the second biological product in the fifth solution, or a quantity related to a concentration of the second biological product in the fifth solution.

61. The method of claim 57, wherein the spectral information for the fifth solution comprises at least one member of the group consisting of an infrared spectrum of the fifth solution, an ultraviolet spectrum of the fifth solution, and a Raman scattering spectrum of the fifth solution.

62. The method of claim 61, further comprising measuring attenuated total reflection of incident infrared light to obtain the infrared spectrum of the fifth solution.

63. The method of claim 53, further comprising measuring the refractive index of the fifth solution by measuring attenuated total reflection of incident infrared light from the fifth solution.

64. The method of claim 1, wherein the biological product is measured at the locations upstream and downstream from the dilution location by measuring different types of spectral information corresponding to the respective first and third solutions.

65. The method of claim 51, wherein the measured values of the second biological product or values derived from the measured values of the second biological product at the upstream and downstream locations are of a different type.

66. The method of claim 51, comprising obtaining the measured values of the second biological product or values derived from the measured values of the second biological product at the upstream and downstream locations using different measurement techniques.

67. The method of claim 51, wherein the fourth solution does not comprise the second biological product.

68. The method of claim 67, wherein the fourth solution does not comprise the first biological product.

69. The method of claim 1, wherein the flowing first solution is received from a purification unit of a biological manufacturing system.

70. The method of claim 69, wherein the purification unit comprises a tangential flow filtration unit.

71. A system, comprising:
a flow channel comprising an inlet;
a fluid reservoir connected to the flow channel at a dilution location;
at least one flow regulator connected between at least one of the fluid reservoir and the dilution location, and the inlet and the dilution location;
a first sensor positioned at an upstream location between the inlet of the flow channel and the dilution location;
a second sensor positioned at a downstream location between an outlet of the flow channel and the dilution location; and
a controller connected to the first and second sensors and to the flow regulator,
wherein the first sensor is configured to measure a biological product in a flowing first solution that enters the inlet;
wherein the fluid reservoir is configured to introduce a second solution into the flow channel at the dilution location to form a flowing third solution;
wherein the second sensor is configured to measure the biological product the flowing third solution; and
wherein the controller is configured to:
determine a relative relationship between measured values of the biological product or values derived from measured values of the biological product at the upstream and downstream locations; and
adjust the at least one flow regulator to control a flow rate of at least one of the first solution and the second solution based on the relative relationship.

72. The system of claim 71, wherein the first sensor is configured to obtain a measured value of a parameter of the first solution.

73. The system of claim 72, wherein the first sensor is a refractive index sensor configured to measure a refractive index of the first solution.

74. The system of claim 72, wherein the first sensor is a conductivity sensor configured to measure a conductivity of the first solution.

75. The system of claim 72, wherein the first sensor is an absorbance sensor configured to measure an absorbance of the first solution.

76. The system of claim 72, wherein the first sensor is a transmittance sensor configured to measure a transmittance of the first solution.

77. The system of claim 72, wherein the first sensor is a reflectance sensor configured to measure a reflectance of the first solution.

78. The system of claim 72, wherein the first sensor is a concentration sensor configured to measure a concentration of the biological product in the first solution.

79. The system of claim 72, wherein the first sensor is configured to measure the value of the parameter at a single wavelength.

80. The system of claim 79, wherein the single wavelength is in an ultraviolet spectral region.

81. The system of claim 79, wherein the single wavelength is in a visible spectral region.

82. The system of claim 79, wherein the single wavelength is in an infrared spectral region.

83. The system of claim 71, wherein the first sensor is configured to obtain a plurality of measured values of the first solution.

84. The system of claim 73, wherein the plurality of measured values comprise spectral information for the first solution at a plurality of wavelengths.

85. The system of claim 74, wherein the controller is configured to analyze the spectral information to determine a value derived from the spectral information.

86. The system of claim 85, wherein the controller is configured to analyze the spectral information by using a calibrated chemometric model to determine the value derived from the spectral information.

87. The system of claim 85, wherein the value derived from the spectral information is a concentration of the biological product in the first solution, or a quantity related to a concentration of the biological product in the first solution.

88. The system of claim 84, wherein the first sensor is configured to obtain an infrared spectrum of the first solution.

89. The system of claim 84, wherein the first sensor is configured to obtain an ultraviolet spectrum of the first solution.

90. The system of claim 84, wherein the first sensor is configured to obtain a Raman scattering spectrum of the first solution.

91. The system of claim 88, wherein the first sensor is configured to measure attenuated total reflection of incident infrared light to obtain the infrared spectrum.

92. The system of claim 73, wherein the first sensor is configured to measure the refractive index of the first solution by measuring attenuated total reflection of incident infrared light from the first solution.

93. The system of claim 71, wherein the second sensor is configured to obtain a measured value of a parameter of the third solution.

94. The system of claim 93, wherein the second sensor is configured to measure at least one member of the group consisting of: a refractive index of the third solution; a conductivity of the third solution; an absorbance of the third solution; a transmittance of the third solution; a reflectance of the third solution; and a concentration of the biological product in the third solution.

95. The system of claim 93, wherein the second sensor is configured to measure the value of the parameter of the third solution at a single wavelength.

96. The system of claim 95, wherein the single wavelength comprises a wavelength in an ultraviolet, visible, or infrared spectral region.

97. The system of claim 71, wherein measuring the second sensor is configured to obtain a plurality of measured values of the third solution.

98. The system of claim 97, wherein the plurality of measured values comprise spectral information for the third solution at a plurality of wavelengths.

99. The system of claim 98, wherein the controller is configured to analyze the spectral information to determine a value derived from the spectral information.

100. The system of claim 99, wherein the controller is configured to analyze the spectral information by using a calibrated chemometric model to determine the value derived from the spectral information.

101. The system of claim 99, wherein the value derived from the spectral information is a concentration of the biological product in the third solution, or a quantity related to a concentration of the biological product in the third solution.

102. The system of claim 98, wherein the second sensor is configured to obtain spectral information comprising at least one member of the group consisting of an infrared spectrum of the third solution, an ultraviolet spectrum of the third solution, and a Raman scattering spectrum of the third solution.

103. The system of claim 102, wherein the second sensor is configured to measure attenuated total reflection of incident infrared light to obtain the infrared spectrum.

104. The system of claim 94, wherein the second sensor is configured to measure the refractive index of the third solution by measuring attenuated total reflection of incident infrared light from the third solution.

105. The system of claim 71, wherein the first and second sensors are configured to measure the biological product in the first and third solutions using different measurement techniques.

106. The system of claim 105, wherein the at least one of a measured value of a parameter of the first solution and spectral information for the first solution comprises a different type of information from the at least one of a measured value of a parameter of the third solution and spectral information for the third solution.

107. The system of claim 105, wherein the at least one of a measured value of a parameter of the first solution and spectral information for the first solution, and the at least one of a measured value of a parameter of the third solution and spectral information for the third solution, are each independently selected from the group consisting of a refractive index; a conductivity; an absorbance; a transmittance; a reflectance; a concentration of the biological product; an infrared spectrum; an ultraviolet spectrum; and a Raman scattering spectrum.

108. The system of claim 71, wherein the first and second sensors are configured to measure the biological product in the first and third solutions using a common measurement technique.

109. The system of claim 108, wherein the at least one of a measured value of a parameter of the first solution and spectral information for the first solution, and the at least one of a measured value of a parameter of the third solution and spectral information for the third solution, are each independently selected from the group consisting of a refractive index; a conductivity; an absorbance; a transmittance; a reflectance; a concentration of the biological product; an infrared spectrum; an ultraviolet spectrum; and a Raman scattering spectrum.

110. The system of claim 71, wherein the second solution does not comprise the biological product.

111. The system of claim 71, wherein the first and second sensors are of a different type.

112. The system of claim 71, wherein the controller is configured to determine a relative relationship between measured values of the biological product or values derived from the measured values of the biological product at the upstream and downstream locations by:
obtaining a first value correlated with a concentration of the biological product or a quantity related to a concentration of the biological product in the first solution;
obtaining a second value correlated with a concentration of the biological product or a quantity related to a concentration of the biological product in the third solution;
calculating a comparative quantity based on the first and second values; and
adjusting the at least one regulator to control the flow rate of the at least one of the first solution and the second solution based on the comparative quantity.

113. The system of claim 112, wherein the controller is configured to calculate the comparative quantity as a ratio of the first and second values.

114. The system of claim 112, wherein the controller is configured to calculate the comparative quantity as a mathematical function of the first and second values.

115. The system of claim 71, wherein the controller is configured to adjust the at least one regulator to control the flow rate of the at least one of the first solution and the second solution until a value of the relative relationship is within a target range of values.

116. The system of claim 112, wherein the controller is configured to adjust the at least one flow regulator to control the flow rate of the at least one of the first solution and the second solution until a value of the comparative quantity is within a target range of values.

117. The system of claim 71, wherein the biological product is a protein.

118. The system of claim 117, wherein the protein is an antibody, an antibody fragment, or comprises a portion of an antibody.

119. The system of claim 71, wherein the biological product in the first solution is a drug substance, and wherein the third solution is a drug product.

120. The system of claim 71, further comprising a purification unit for use in a biological manufacturing system, wherein the purification unit is in fluid communication with the inlet.

121. The system of claim 120, wherein the purification unit comprises a tangential flow filtration unit.

122. The method of claim 2, wherein the measured value of the parameter of the first solution is an osmolality of the first solution.

123. The method of claim 23, wherein the measured value of the parameter of the third solution is an osmolality of the third solution.

124. The method of claim 1, wherein measuring the biological product at the upstream location comprises obtaining an osmolality value for the first solution, and wherein measuring the biological product at the downstream location comprises obtaining an osmolality value for the third solution.

125. The method of claim 124, comprising adjusting the flow rate of the second solution based on the relative relationship.

126. The method of claim 124, wherein the relative relationship is a ratio of the osmolality values for the first and third solutions.

127. A method, comprising:
receiving a flowing first solution comprising a biological product and directing the flowing first solution along a flow path, wherein the flow path comprises a dilution location at which a dilution apparatus is in fluid communication with the flow path;
introducing a second solution into the flowing first solution at the dilution location to form a flowing third solution;
measuring a value of an attribute of the second solution at a location upstream from the dilution location;
measuring a value of an attribute of the third solution at a location downstream from the dilution location;
determining a relative relationship between the measured attribute values of the second and third solutions; and
adjusting a flow rate of the second solution based on the relative relationship.

128. The method of claim 127, wherein the measured value of the attribute of the second solution is an osmolality of the second solution.

129. The method of claim 127, wherein the measured value of the attribute of the second solution is a refractive index of the second solution.

130. The method of claim 127, wherein the measured value of the attribute of the second solution is a conductivity of the second solution.

131. The method of claim 127, wherein the measured value of the attribute of the second solution is an absorbance of the second solution.

132. The method of claim 127, wherein the measured value of the attribute of the second solution is a transmittance of the second solution.

133. The method of claim 127, wherein the measured value of the attribute of the second solution is a reflectance of the second solution.

134. The method of claim 127, wherein the measured value of the attribute of the second solution is measured at a single wavelength.

135. The method of claim 127, wherein measuring the value of the attribute of the second solution comprises obtaining a plurality of measured values of the second solution.

136. The method of claim 135, wherein the plurality of measured values comprise spectral information for the second solution at a plurality of wavelengths.

137. The method of claim 136, further comprising analyzing the spectral information to determine the value of the attribute of the second solution.

138. The method of claim 137, wherein analyzing the spectral information comprises using a calibrated chemometric model to determine the value of the attribute of the second solution.

139. The method of claim 138, wherein the value of the attribute is an osmolality of the second solution.

140. The method of claim 136, wherein the spectral information comprises a Raman scattering spectrum of the second solution.

141. The method of claim 136, wherein the spectral information comprises an infrared spectrum of the second solution.

142. The method of claim 136, wherein the spectral information comprises an ultraviolet spectrum of the second solution.

143. The method of claim 127, wherein the measured value of the attribute of the third solution is an osmolality of the third solution.

144. The method of claim 127, wherein the measured value of the attribute of the second solution is an osmolality of the second solution, and the measured value of the attribute of the third solution is an osmolality of the third solution.

145. The method of claim 127, wherein the measured value of the attribute of the third solution comprises at least one member of the group consisting of: a refractive index of the third solution; a conductivity of the third solution; an absorbance of the third solution; a transmittance of the third solution; and a reflectance of the third solution.

146. The method of claim 127, wherein the measured value of the attribute of the third solution is measured at a single wavelength.

147. The method of claim 127, wherein measuring the value of the attribute of the third solution comprises obtaining a plurality of measured values of the third solution.

148. The method of claim 147, wherein the plurality of measured values comprise spectral information for the third solution at a plurality of wavelengths.

149. The method of claim 148, further comprising analyzing the spectral information to determine the value of the attribute of the third solution.

150. The method of claim 149, wherein analyzing the spectral information comprises using a calibrated chemometric model to determine the value of the attribute of the third solution.

151. The method of claim 150, wherein the value of the attribute is an osmolality of the third solution.

152. The method of claim 148, wherein the spectral information comprises a Raman scattering spectrum of the third solution.

153. The method of claim 148, wherein the spectral information comprises an infrared spectrum of the third solution.

154. The method of claim 148, wherein the spectral information comprises an ultraviolet spectrum of the third solution.

155. The method of claim 127, wherein the values of the attributes of the second and third solutions are measured using different measurement techniques.

156. The method of claim 155, wherein the attributes of the second and third solutions for which values are measured are different.

157. The method of claim 127, wherein the second solution does not comprise the biological product.

158. The method of claim 127, wherein a flow rate of the flowing first solution along the flow path is less than 2 mL/min.

159. The method of claim 127, wherein determining a relative relationship between the measured attribute values of the second and third solutions comprises calculating a comparative quantity between the measured attribute values.

160. The method of claim 159, wherein the comparative quantity is a ratio of the measured attribute values.

161. The method of claim 159, wherein the comparative quantity is a mathematical function of the measured attribute values.

162. The method of claim 127, further comprising adjusting the flow rate of the second solution until a value of the relative relationship is within a target range of values.

163. The method of claim 159, further comprising adjusting the flow rate of the second solution until a value of the comparative quantity is within a target range of values.

164. The method of claim 127, wherein the biological product is a protein.

165. The method of claim 164, wherein the protein is an antibody, an antibody fragment, or comprises a portion of an antibody.

166. The method of claim 127, wherein the biological product in the first solution is a drug substance, and wherein the third solution is a drug product.

167. A system, comprising:
a flow channel comprising an inlet;
a fluid reservoir connected to the flow channel at a dilution location;
at least one flow regulator connected between the fluid reservoir and the dilution location;
a first sensor positioned between the fluid reservoir and the dilution location;
a second sensor positioned at a downstream location between an outlet of the flow channel and the dilution location; and
a controller connected to the first and second sensors and to the flow regulator,
wherein the flow channel is configured to receive a flowing first solution comprising a biological product through the inlet;
wherein the fluid reservoir is configured to introduce a second solution into the flow channel at the dilution location to form a flowing third solution;
wherein the first sensor is configured to measure an attribute value of the second solution;
wherein the second sensor is configured to measure an attribute value of the third solution; and
wherein the controller is configured to:
determine a relative relationship between the measured attribute values of the second and third solutions; and
adjust the at least one flow regulator to control a flow rate of the second solution based on the relative relationship.

168. The system of claim 167, wherein the first sensor is a Raman scattering sensor configured to measure Raman scattered light from the second solution.

169. The system of claim 167, wherein the first sensor is a refractive index sensor configured to measure a refractive index of the second solution.

170. The system of claim 167, wherein the first sensor is a conductivity sensor configured to measure a conductivity of the second solution.

171. The system of claim 167, wherein the first sensor is an absorbance sensor configured to measure an absorbance of the second solution.

172. The system of claim 167, wherein the first sensor is a transmittance sensor configured to measure a transmittance of the second solution.

173. The system of claim 167, wherein the first sensor is a reflectance sensor configured to measure a reflectance of the second solution.

174. The system of claim 167, wherein the first sensor is configured to obtain a plurality of measured values of the second solution.

175. The system of claim 174, wherein the plurality of measured values comprises spectral information for the second solution at a plurality of wavelengths.

176. The system of claim 175, wherein the controller is configured to analyze the spectral information to determine the measured attribute value of the second solution.

177. The system of claim 176, wherein the controller is configured to analyze the spectral information by using a calibrated chemometric model to determine the measured attribute value of the second solution from the spectral information.

178. The system of claim 175, wherein the first sensor is configured to obtain a Raman scattering spectrum of the second solution.

179. The system of claim 167, wherein the second sensor is a Raman scattering sensor configured to measure Raman scattered light from the third solution.

180. The system of claim 167, wherein the second sensor comprises at least one member of the group consisting of: a refractive index sensor configured to measure a refractive index of the third solution; a conductivity sensor configured to measure a conductivity of the third solution; an absorbance sensor configured to measure an absorbance of the third solution; a transmittance sensor configured to measure a transmittance of the third solution; and a reflectance sensor configured to measure a reflectance of the third solution.

181. The system of claim 167, wherein the second sensor is configured to obtain a plurality of measured values of the third solution.

182. The system of claim 181, wherein the plurality of measured values comprise spectral information for the third solution at a plurality of wavelengths.

183. The system of claim 182, wherein the controller is configured to analyze the spectral information to determine the measured attribute value of the third solution from the spectral information.

184. The system of claim 183, wherein the controller is configured to analyze the spectral information by using a calibrated chemometric model to determine the measured attribute value of the third solution.

185. The system of claim 182, wherein the second sensor is configured to obtain a Raman scattering spectrum of the third solution.

186. The system of claim 167, wherein the first and second sensors are configured to measure the attribute values of the second and third solutions using different measurement techniques.

187. The system of claim 186, wherein the attributes of the second and third solutions for which the values are measured are different.

188. The system of claim 167, wherein the second solution does not comprise the biological product.

189. The system of claim 67, wherein the first and second sensors are of a different type.

190. The system of claim 67, wherein the controller is configured to:
calculate a comparative quantity based on the measured attribute values of the second and third solutions; and
adjust the at least one regulator to control the flow rate of the second solution based on the comparative quantity.

191. The system of claim 190, wherein the controller is configured to calculate the comparative quantity as a ratio of the measured attribute values.

192. The system of claim 190, wherein the controller is configured to calculate the comparative quantity as a mathematical function of the measured attribute values.

193. The system of claim 167, wherein the controller is configured to adjust the at least one regulator to control the flow rate of the second solution until a value of the relative relationship is within a target range of values.

194. The system of claim 190, wherein the controller is configured to adjust the at least one flow regulator to control the flow rate of the second solution until a value of the comparative quantity is within a target range of values.

195. The system of claim 167, wherein the biological product is a protein.

196. The system of claim 195, wherein the protein is an antibody, an antibody fragment, or comprises a portion of an antibody.

197. The system of claim 167, wherein the biological product in the first solution is a drug substance, and wherein the third solution is a drug product.

198. The system of claim 167, further comprising a purification unit, wherein the purification unit is in fluid communication with the inlet.

199. The system of claim 198, wherein the purification unit comprises a tangential flow filtration unit.
